(19) United States
(12) Patent Application Publication
(10) Pub. No.: US 2019/0091162 A1
(43) Pub. Date: Mar. 28, 2019

(54) POLYMER-LIPID HYBRID NANOPARTICLES OF CAPECITABINE UTILIZING MICROMIXING AND CAPECITABINE AMPHIPHILIC PROPERTIES

(71) Applicant: Jingjie PTM BioLab (Hangzhou) Co., Ltd., Hangzhou (CN)

(72) Inventor: Zhongyi CHENG, Hangzhou (CN)

(73) Assignee: Jingjie PTM BioLab (Hangzhou) Co., Ltd., Hangzhou (CN)

(21) Appl. No.: 16/137,155

(22) Filed: Sep. 20, 2018

Related U.S. Application Data
(60) Provisional application No. 62/561,744, filed on Sep. 22, 2017.

(51) Int. Cl. A61K 9/51; A61K 31/7068; A61K 45/06; A61K 9/00

(52) U.S. Cl. CPC A61K 9/5123; A61K 31/7068; A61K 9/5192; A61K 45/06; A61K 9/0019; A61K 9/5146

(57) ABSTRACT

The present disclosure includes compositions and methods of making a nanoparticle composition comprising a phospholipids core comprising one or more lipids and one or more active agents, and at least one layer of one or more polymers on the surface of the phospholipids core; more specifically, the disclosure relates to the use of capecitabine (N4-pentyloxycarbonyl-5-deoxy-5-fluoro-cytidine, CAP) within such a lipid-polymer nanoparticle formulation for optimizing pharmaceutical properties of capecitabine for the treatment of cancer.

The present application claims priority to U.S. Provisional Patent Application No. 62/561,744, filed Sep. 22, 2017, the entirety of which is hereby incorporated by reference.

### TECHNICAL FIELD OF THE DISCLOSURE

The present disclosure relates in general to nanoparticles comprising a phospholipids core comprising one or more lipids and one or more active agents and at least one layer of one or more polymers on the surface of the phospholipids core. More specifically, the disclosure relates to the use of capecitabine (N4-pentyloxycarbonyl-5-deoxy-5-fluoro-cytidine, CAP) within such a lipid-polymer nanoparticle formulation for reducing side effects of capecitabine for the treatment of cancer.

### BACKGROUND OF THE DISCLOSURE

Without limiting the scope of the disclosure, its background is described in connection with the delivery of active pharmaceutical agents, more specifically capecitabine (N4-pentyloxycarbonyl-5-deoxy-5-fluoro-cytidine, CAP) or its metabolites. While many US patent publications are said to provide nanoparticle formulation of cancer chemotherapeutic drugs, none of them is directly linked to lipid or polymer delivery of capecitabine, specifically a polymer-lipid hybrid delivery of capecitabine or its metabolites.

### SUMMARY OF THE DISCLOSURE

Some embodiments described herein are nanoparticle compositions comprising a nanoparticle core comprising one or more phospholipids and at least one active ingredient comprising capecitabine or its active metabolites and at least one layer of one or more polymers on a surface of the phospholipids core. In some embodiments of the nanoparticle compositions, one or more of the phospholipids is neutral or positively charged.

In some embodiments, the one or more phospholipids present in the nanoparticle composition comprises at least one of 1,2-Didecanoyl-sn-glycero-3-phosphocholine (DDPC), 1,2-dilauroyl-sn-glycero-3-phosphoethanolamine (DLPE), dimyristoyl phosphatidylcholine (DMPC), dimyristoyl phosphatidylglycerol (DMPG), 1,2-dimyristoyl-sn-glycero-3-phosphoethanolamine (DMPE-PEG), 1-palmitoyl-2-myristoyl-sn-glycero-3-phosphocholine (PMPC), 1,2-dipalmitoyl-sn-glycero-3-phospho-(1′-rac-glycerol) (DPPG), 1,2-dipalmitoyl-sn-glycero-3-phosphocholine (DPPC), 1,2-dipalmitoyl-sn-glycero-3-phosphoethanolamine (DPPE-PEG), 1,2-dipalmitoyl-sn-glycero-3-phosphate (sodium salt) (DPPA), 1-palmitoyl-2-oleoyl-sn-glycero-3-phosphocholine (POPC), 1-palmitoyl-2-stearoyl-sn-glycero-3-phosphocholine (PSOC), 1-stearoyl-2-palmitoyl-sn-glycero-3-phosphocholine (SPPC), 1,2-distearoyl-sn-glycero-3-phosphoethanolamine-N-[methoxy(polyethylene glycol)-2000] (DEPE-PEG), 1,2-distearoyl-sn-glycero-3-phosphoethanolamine-N-[dibenzocyclooctyl(polyethylene glycol)-2000] (DSPE-PEG), L-α-phosphatidylcholine (L-α-PC), 1,2-dilinoleoyl-sn-glycero-3-phosphocholine (DLPC), 1,2-dioleoyl-sn-glycero-3-phospho-(1′-rac-glycerol) (DOPG), 1,2-distearoyl-sn-glycero-3-phospho-(1′-rac-glycerol) (DSPG), 1,2-Distearoyl-3-trimethylammonium-propane (DSTAP), 1,2-dioleoyl-3-trimethylammonium-propane (DOTAP), 1,2-dioleoyl-sn-glycero-3-phosphate (DOPA), 1,2-dioleoyl-sn-glycero-3-phosphoethanolamine (DOPE), 1-stearoyl-2-oleoyl-sn-glycero-3-phosphocholine (SOPC), or 1,2-dioleoyl-sn-glycero-3-phosphocholine (DOPC) or combinations thereof.

In some embodiments, the one or more phospholipids present in the nanoparticle composition comprises at least one of poly(lactic-co-glycolic acid) (PLGA) or its PEGylated form PEG-PLGA, polylactic acid (PLA) or its PEGylated form PEG-PLA, polyglycolic acid (PGA) or its PEGylated form PEG-PGA, poly-L-lactide-co-ε-caprolactone (PLCL) or its PEGylated form PEG-PLCL, Hyaluronic acid (HA), polyacrylic acid (PAA) or PEG-PAA, polyphosphate (polyP), poly(acrylic acid-co-maleic acid), poly(butylene succinate), poly(alkyl cyanoacrylate) (PAC) or its PEGylated form PEG-PAC or combinations thereof.

In some embodiments, the nanoparticle composition further comprises an active agent comprising an anti-cancer drug, an antibiotic, an antiviral, an antifungal, an antihelminthic, a nutrient, a small molecule, a siRNA, an antioxidant, an antibody, or a radioisotope, or combinations thereof.

In some embodiments, the one or more phospholipids present in the nanoparticle composition comprises 1,2-dipalmitoyl-sn-glycero-3-phosphocholine (DPPC), 1,2-dipalmitoyl-sn-glycero-3-phosphoethanolamine (DPPE-PEG), L-α-phosphatidylcholine (L-α-PC), 1,2-dilinoleoyl-sn-glycero-3-phosphocholine (DLPC), or 1,2-dioleoyl-3-trimethylammonium-propane (DOTAP) or combinations thereof.

In some embodiments, the one or more phospholipids present in the nanoparticle composition is 1,2-dipalmitoyl-sn-glycero-3-phosphocholine (DPPC) and 1,2-dipalmitoyl-sn-glycero-3-phosphoethanolamine (DPPE-PEG). In some embodiments, the one or more phospholipids present in the nanoparticle composition is L-α-phosphatidylcholine (L-α-PC) and 1,2-dipalmitoyl-sn-glycero-3-phosphoethanolamine (DPPE-PEG). In some embodiments, the one or more phospholipids present in the nanoparticle composition is 1,2-dioleoyl-3-trimethylammonium-propane (DOTAP).

In some embodiments, the one or more phospholipids present in the nanoparticle composition is 1,2-dioleoyl-3-trimethylammonium-propane (DOTAP) and the polymer is polyphosphate (polyP). In some embodiments, the one or more phospholipids present in the nanoparticle composition is 1,2-dioleoyl-3-trimethylammonium-propane (DOTAP) and the polymer is PEG polyacrylic acid (PAA).

In some embodiments, a molar ratio of the lipid(s) to a PEGylated lipid(s) in the nanoparticle composition is about 100:0 to about 50:50. In some embodiments, a molar ratio of a saturated lipid(s) to an unsaturated lipid(s) in the nanoparticle composition is about 100:0 to about 25:75. In some embodiments, a molar ratio of capecitabine to lipid(s) in the nanoparticle composition is about 90:10 to about 10:90. In some embodiments, a molar ratio of lipid(s) to polymer in the nanoparticle composition is about 100:0 to about 10:80. In some embodiments, a molar ratio of capecitabine to polymer in the nanoparticle composition is about 100:0 to about 10:90. In some embodiments, the nanoparticle composition exhibits a zeta potential of from about −80 mV to about 80 mV.

In some embodiments, a surface of the nanoparticle core in the nanoparticle composition is neutral. In some embodiments, the surface of the nanoparticle core is positively charged or negatively charged.

In some embodiments, the nanoparticle composition further comprises at least one targeting agent, wherein the targeting agent selectively targets the nanoparticle to diseased tissue/cells, thereby minimizing whole body dose. In some embodiments, the nanoparticle composition further comprises at least one targeting agent, wherein the targeting agent comprises an antibody or functional fragment thereof, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

In some embodiments, the nanoparticles of the nanoparticle composition have a size of about 10 nm to about 200 nm. In some embodiments, a drug load of capecitabine in the nanoparticle composition is about 2% to about 90% by weight of the composition.

In some embodiments, the structure of the nanoparticle composition provides sustained release of capecitabine or its active metabolites when provided to a subject. In some embodiments, a bioavailability of the active agent is increased, one or more side effects such as nausea, vomiting, dermatitis, bone-marrow depression, cardiotoxicity and diarrhea is reduced, and the active agent is released in a sustained manner. In some embodiments, the nanoparticle composition is adapted for intramuscular, subcutaneous, intravascular, or intravenous administration.

Some embodiments described herein are methods of forming a nanoparticle composition comprising:
a). forming an organic phase by combining one or more phospholipids, one or more solvents and at least one of capecitabine or its metabolites;
b). forming a lipid aqueous phase by combining one or more targeting agents with water;
c). mixing the organic phase with the aqueous phase, whereby self-assembly of micelles occurs, thereby forming a suspension;
d). spray drying or freeze drying the suspension; and
e). mixing solution with one or more polymers with the micelles, whereby layer-by-layer polymer deposition occurs and wherein the capecitabine or its metabolites nanoparticles provide sustained release of active components when provided to a subject.

In some embodiments, the manufactured nanoparticles are produced in a uniform size with uniform physicochemical properties.

Some embodiments described herein are methods for treating a patient suspected of being afflicted with a disease comprising administering the nanoparticle composition described herein to a subject in need thereof. In some embodiments, administering the nanoparticles comprises administering the nanoparticles by intramuscular, subcutaneous, intravascular, or intravenous administration. In some embodiments, the disease to be treated is selected from the group consisting of oncologic, neurologic, and metabolic diseases. In some embodiments, the disease is selected from the group consisting of Parkinson's disease, Alzheimer's disease, multiple sclerosis, ALS, sequel, behavioral and cognitive disorders, autism spectrum, depression, and neoplastic disease. In some embodiments, following administration of the nanoparticle composition, the active agent is released in a sustained manner.

Some embodiments described herein are pharmaceutical compositions comprising the nanoparticle composition described herein and a pharmaceutically acceptable carrier. In some embodiments, administration of the composition to a subject reduces one or more side effects comprising nausea, vomiting, dermatitis, bone-marrow depression, cardiotoxicity or diarrhea or a combination thereof compared to administration of capecitabine that is not formulated in the nanoparticle composition.

Some embodiments described herein are methods for treating a subject suspected of having cancer comprising:

identifying a subject suspected of having a cancer; and

administering an effective amount of the nanoparticle compositions described herein to the subject, wherein administration of the composition reduces one or more side effects comprising nausea, vomiting, dermatitis, bone-marrow depression, cardiotoxicity or diarrhea or a combination thereof when provided to a subject compared to administration of capecitabine that is not formulated in the nanoparticle composition.

In some embodiments, the treated cancer is a breast cancer, colorectal cancer, or a pancreatic cancer.

### BRIEF DESCRIPTION OF THE DRAWINGS

For a more complete understanding of the features and advantages of the present disclosure, reference is now made to the detailed description of the disclosure along with the accompanying figures and in which:

FIG. 1. Illustration of polymer-lipid nanoparticles encapsulating capecitabine.

FIG. 2. Geometry of the central mixing part of the MIVM.

FIG. 3. Capecitabine molecular structure.

FIG. 4. Solubility curve of CAP in DI water measured by DLS with low laser intensity (left) and medium laser intensity (right).

FIG. 5. Correlation functions at different CAP concentrations. (A) water, (B) 0.01 mg/ml CAP in DI water, (C) 0.1 mg/ml CAP in DI water, (D) 0.5 mg/ml CAP in DI water, (E) 2.5 mg/ml CAP in DI water, (F) 5 mg/ml CAP in DI water, (G) 10 mg/ml CAP in DI water, (H) 20 mg/ml CAP in DI water.

FIG. 6. Structure of the lipid-CAP micelle.

FIG. 7. DLS data of micelle size distribution (A) CAP: DPPC: DPPE-PEG (30:40:30), (B) CAP: DPPE-PEG (30:50). The X-axis is the diameter of the nanoparticles and y-axis is the relative volume of the material mass forming the size of the nanoparticles.

FIG. 8. CAP release from different micelle formulations. The X-axis is time and y-axis is percentage of accumulative release of CAP from the nanoparticles.

FIG. 9. Size distribution of micelles of CAP, L-α-PC, and DPPE-PEG. The X-axis is the diameter of the nanoparticles and y-axis is the relative volume of the material mass forming the size of the nanoparticles.

FIG. 10. Release of CAP from the L-α-PC-DPPE-PEG micelles compared to pure CAP. The X-axis is time and y-axis is percentage of accumulative release of CAP from the nanoparticles.

FIG. 11. DOTAP-CAP micelle size distribution. The X-axis is the diameter of the nanoparticles and y-axis is the relative volume of the material mass forming the size of the nanoparticles

FIG. 12. Release profile of CAP from DOTAP-CAP micelles. The X-axis is time and y-axis is percentage of accumulative release of CAP from the nanoparticles.

FIG. 13. Structure of the polyP-DOTAP-CAP nanoparticle.

FIG. 14. Left: size distribution of DOTAP-CAP micelles before adding polyP. Right: size distribution of polyP-DOTAP-CAP nanoparticles after adding polyP. The X-axis is the diameter of the nanoparticles and y-axis is the relative volume of the material mass forming the size of the nanoparticles.

FIG. 15. Zeta potential (left) and size (right) of polyP-DOTAP-CAP nanoparticles monitored over five days. The X-axis indicates the time points of the measurements and y-axis is zeta-potential of the particles.

FIG. 16. Release of CAP from polyP-DOTAP-CAP nanoparticles compared with pure CAP micelles. The X-axis is time and y-axis is percentage of accumulative release of CAP from the nanoparticles.

FIG. 17. Structure PEG-PAA-DOTAP-CAP nanoparticles.

FIG. 18. Size distribution of PEG-PAA-DOTAP-CAP nanoparticles. The X-axis is the diameter of the nanoparticles and y-axis is the relative volume of the material mass forming the size of the nanoparticles.

FIG. 19. Zeta potential (left) and size (right) of PEG-PAA-DOTAP-CAP nanoparticles compared to polyP-DOTAP-CAP for 5 days. The X-axis indicates the time points of the measurements and y-axis is zeta-potential of the particles.

FIG. 20. Release profile of DOTAP/CAP and PEG-PAA hybrid particles. The X-axis is time and y-axis is percentage of accumulative release of CAP from the nanoparticles.

### DETAILED DESCRIPTION OF THE DISCLOSURE

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not limit the scope of the disclosure.

### Genral Terms

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims. All publications and patent applications mentioned in the specification are indicative of the level of skill of those skilled in the art to which this disclosure pertains.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific embodiments of the disclosure, but their usage does not delimit the disclosure, except as outlined in the claims. The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. In certain embodiments, the present disclosure may also include methods and compositions in which the transition phrase “consisting essentially of” or “consisting of” may also be used.

The terms “active ingredient” or “active pharmaceutical ingredient” as used herein refer to a pharmaceutical agent, active ingredient, compound, or substance, or mixtures thereof. The active ingredient may be in the form of pharmaceutically acceptable uncharged or charged molecules, molecular complexes, solvates, or anhydrates thereof, and, if relevant, single isomers, enantiomers, racemic mixtures, or mixtures thereof. Furthermore, the active pharmaceutical ingredient may be in any of its crystalline, polymorphous, semi-crystalline, amorphous, or polyamorphous forms, or mixtures thereof.

The terms “active pharmaceutical ingredient load” or “drug load” as used herein refers to the quantity (mass) or weight percentage (wt %) of the active pharmaceutical ingredient comprised in the nanoparticle compositions described herein.

As used herein, the term “capecitabine” or “CAP” refers to any pharmacologically active form or prodrug form of capecitabine, including any salt, crystalline, polymorphous, semi-crystalline, amorphous, or polyamorphous forms thereof. Further,

As used herein, the phrase “active metabolite of capecitabine” or the phrase “capecitabine or its active metabolites” is intended to include any metabolite of capecitabine that is generated by the body of a subject following administration of capecitabine via parenteral or non-parenteral means. The active metabolite of capecitabine can include, for example, 5′-deoxy-5-fluorocytidine (5′-DFCR), 5-fluoro-6-hydroxycytosine (5-FCOH), 5′-deoxy-5-fluorouridine (5′-DFUR), 5-fluorouracil (5-FU), 2′-β-D-glucuronide of 5′-deoxy-5-fluorocytidine (5′-DFCR-G), 5-fluorocytosine (5-FC), fluoroacetate (FAC), α-fluoro-β-alanine (FBAL), 5,6-dihydro-5-fluorouracil (5-FUH2), α-fluoro-β-ureidopropionic acid (FUPA), 2-fluoro-3-hydroxypropionic Acid (FHPA), or N-carboxy-α-fluoro-β-alanine (CFBAL) or combinations thereof. The metabolism of capecitabine to its various metabolites are known in the art (see, e.g., Desmoulin et al., Drug Metabolism and Disposition, 30(11), pp. 1221-1229 (2002), which is incorporated by reference herein).

The term “treating” refers to administering a therapy in an amount, manner, or mode effective (e.g., a therapeutic effect) to improve a condition, symptom, disorder, or parameter associated with a disorder, or a likelihood thereof.

As used herein, the “effective amount” refers to the amount necessary to elicit the desired biological response. As will be appreciated by those of ordinary skill in this art, the effective amount of nanoparticle compositions of capecitabine may vary depending on such factors as the desired biological endpoint, the drug to be delivered, the target tissue, the route of administration, etc. For example, the effective amount of the nanoparticle containing at least capecitabine for treating cancer might be the amount that results in a reduction in tumor size by a desired amount over a desired period of time. Additional factors, which may be taken into account, include the severity of the disease state, age, weight and gender of the patient being treated, diet, time and frequency of administration, drug combinations, reaction sensitivities, and tolerance/response to therapy.

The phrase “enhanced bioavailability,” “improved bioavailability,” or “better bioavailability” as used herein refers to the increased proportion of an active pharmaceutical ingredient that enters the systemic circulation when introduced into the body as compared to a reference active pharmaceutical's bioavailability. Bioavailability can be determined by comparing the rate and extent of absorption of a drug with a reference drug when administered at the same molar dose of the active therapeutic ingredient under similar experimental conditions in either a single dose or multiple doses. Typical pharmacokinetic parameters can be used to demonstrate enhanced bioavailability compared to the reference drug.

The term “substantially” as used herein means to a great or significant extent, but not completely.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

### Nanoparticle Compositions of Capecitabine

The treatment of cancer is limited by the side effects of the anti-cancer drugs. Chemotherapy is among the limited available options for the treatment of advanced cancers. However, increasing evidences of drug resistance and non-specific toxicity of these agents such as capecitabine limits their therapeutic outcomes. To overcome this problem, it is important to deliver the drug at the site of cancer in the body in the right amount. A novel way to approach this problem is through targeted drug delivery system, which preferentially delivers the drug to the site of cancer. As described herein, this approach is through lipid nanoparticles or lipid/polymer hybrid nanoparticles containing an anti-cancer therapeutic (e.g., capecitabine). In certain embodiment, targeting molecules (e.g., antibodies) that recognize the cancer cells and direct the drug containing tiny spherical nanoparticles to the cancer cells are used. Therefore, in certain embodiments, at least one targeting agent is attached to the nanoparticles, wherein the targeting agent comprises an antibody or functional fragment thereof that is capable of recognizing a target antigen, The targeting agents may be attached by insertion of hetero/homo bifunctional spacer capable of reacting with amines of lipids and targeting moieties.

Capecitabine (N4-pentyloxycarbonyl-5-deoxy-5-fluoro-cytidine, CAP) is a popular prodrug that is used to treat colorectal cancer. In the target tissue, CAP is converted by enzymes from 5′-deoxy-5-fluorouridine to 5-fluorouracil, an active metabolite. Despite its effectiveness in treating various cancers, i.e. colorectal cancer, CAP exhibits short drug half-life. By itself, the drug is eliminated from the body within 0.5-1 hours and thus, requires high dose (150 mg/m², twice a day). Large dosage can lead to more side effects such as nausea, vomiting, dermatitis, bone-marrow depression, cardiotoxicity and diarrhea. Therefore, much effort has been made to design a sustained release system of delivery as well as target specific cancer sites. As described herein, nanoparticle formulations according to the embodiments described herein provide an option to solve these problematic issues.

Accordingly, the present disclosure provides compositions and methods of producing stable nanoparticles of capecitabine with well-controlled physicochemical properties such as size and surface properties. The present inventors discovered that advantages of nanoparticle compositions described herein are high bioavailability, sustained release, low in vivo clearance, and reduced side effects, Thus, the commercial potential of the nanoparticle compositions described herein are enormous due to the improved bioavailability, sustained release, and reduced side effects,

Some embodiments of the nanoparticle compositions described herein comprise a phospholipid core comprising one or more lipids and one or more active agents comprising capecitabine or its metabolites. In some embodiments, the lipids comprise at least one of 1,2-Didecanoyl-sn-glycero-3-phosphocholine (DDPC), 1,2-dilauroyl-sn-glycero-3-phosphoethanolamine (DLPE), dimyristoyl phosphatidylcholine (DMPC), dimyristoyl phosphatidylglycerol (DMPG), 1,2-dimyristoyl-sn-glycero-3-phosphoethanolamine (DMPE-PEG), 1-palmitoyl-2-myristoyl-sn-glycero-3-phosphocholine (PMPC), 1,2-dipalmitoyl-sn-glycero-3-phospho-(1′-rac-glycerol) (DPPG), 1,2-dipalmitoyl-sn-glycero-3-phosphocholine (DPPC), 1,2-dipalmitoyl-sn-glycero-3-phosphoethanolamine (DPPE-PEG), 1,2-dipalmitoyl-sn-glycero-3-phosphate (sodium salt) (DPPA), 1-palmitoyl-2-oleoyl-sn-glycero-3-phosphocholine (POPC), 1-palmitoyl-2-stearoyl-sn-glycero-3-phosphocholine (PSOC), 1-stearoyl-2-palmitoyl-sn-glycero-3-phosphocholine (SPPC), 1,2-distearoyl-sn-glycero-3-phosphoethanolamine-N-[methoxy(polyethylene glycol)-2000] (DEPE-PEG), 1,2-distearoyl-sn-glycero-3-phosphoethanolamine-N-[dibenzocyclooctyl(polyethylene glycol)-2000] (DSPE-PEG), L-α-phosphatidylcholine (L-α-PC), 1,2-dilinoleoyl-sn-glycero-3-phosphocholine (DLPC), 1,2-dioleoyl-sn-glycero-3-phospho-(1′-rac-glycerol) (DOPG), 1,2-distearoyl-sn-glycero-3-phospho-(1′-rac-glycerol) (DSPG), 1,2-Distearoyl-3-trimethylammonium-propane (DSTAP), 1,2-dioleoyl-3-trimethylammonium-propane (DOTAP), 1,2-dioleoyl-sn-glycero-3-phosphate (DOPA), 1,2-dioleoyl-sn-glycero-3-phosphoethanolamine (DOPE), 1-stearoyl-2-oleoyl-sn-glycero-3-phosphocholine (SOPC), or 1,2-dioleoyl-sn-glycero-3-phosphocholine (DOPC) or combinations thereof. In some embodiments, the one or more lipids comprise 1,2-dipalmitoyl-sn-glycero-3-phosphocholine (DPPC). In some embodiments, the one or more lipids comprise 1,2-dipalmitoyl-sn-glycero-3-phosphoethanolamine (DPPE-PEG). In some embodiments, the one or more lipids comprise L-α-phosphatidylcholine (L-α-PC). In some embodiments, the one or more lipids comprise 1,2-dioleoyl-3-trimethylammonium-propane (DOTAP).

In some embodiments, the nanoparticle composition has a molar ratio of lipid to PEGylated lipid of about 100:0 to about 20:80. In some embodiments, the molar ratio of the lipid to the PEGylated lipid is about 100:0.01 to about 50:50. Thus, in some embodiments, the molar ratio of lipid to PEGylated lipid is about 100:0, about 95:5, about 90:10, about 80:20, about 70:30, about 50:50, about 60:40, about 40:60, about 30:70, or about 20:80. In some embodiments, the molar ratio of total lipid to PEGylated lipid is about 100:0 (i.e., the nanoparticle composition does not contain any or substantially no pegylated lipid). In some embodiments, the molar ratio of lipid to pegylated lipid is about 60:40. In some embodiments, the molar ratio of lipid to pegylated lipid is about 30:70.

In some embodiments, the nanoparticle composition has a molar ratio of saturated lipid to unsaturated lipid of about 100:0 to about 10:90. In some embodiments, the molar ratio of saturated lipid to unsaturated lipid is about 100:0.01 to about 25:75. Thus, in some embodiments, the molar ratio of saturated lipid to unsaturated lipid is about 100:0, about 95:5, about 90:10, about 80:20, about 70:30, about 50:50, about 60:40, about 40:60, about 30:70, about 20:80, or about 10:90.

In some embodiments, the one or more active agents comprise capecitabine (N4-pentyloxycarbonyl-5-deoxy-5-fluoro-cytidine, CAP) or its metabolites. In some embodiments, the one or more active agents comprise an active metabolite of capecitabine. In some embodiments, the active metabolite of capecitabine comprises 5′-deoxy-5-fluorocytidine (5′-DFCR), 5-fluoro-6-hydroxycytosine (5-FCOH), 5′-deoxy-5-fluorouridine (5′-DFUR), 5-fluorouracil (5-FU), 2′-β-D-glucuronide of 5′-deoxy-5-fluorocytidine (5′-DFCR-G), 5-fluorocytosine (5-FC), fluoroacetate (FAC), α-fluoro-β-alanine (FBAL), 5,6-dihydro-5-fluorouracil (5-FUH2), α-fluoro-β-ureidopropionic acid (FUPA), 2-fluoro-3-hydroxypropionic Acid (FHPA), or N-carboxy-α-fluoro-β-alanine (CFBAL) or combinations thereof. In some embodiments, the active metabolite of capecitabine comprises 5-5-fluorouracil, 5′-deoxy-5-fluorocytidine or 5′-deoxy-5-fluorouridine, or 2′-β-D-glucuronide of 5′-deoxy-5-fluorocytidine or combinations thereof.

In some embodiments, the active agent further comprises at least one anti-cancer drug; and/or be selected from at least one of an anti-cancer drug, an antibiotic, an antiviral, an antifungal, an antihelminthic, a nutrient, a small molecule, a siRNA, an antioxidant, and an antibody. In certain aspects, the nanoparticle composition has high bioavailability. In some embodiments, the active agent comprises a radioisotope. In some embodiments, the one or more active agents comprise a water-insoluble dye; and/or a metal nanoparticle, to be used as contrast agents for MRI; and/or be selected from Nile red, iron, and platinum.

In some embodiments, the drug load of active ingredient (e.g., capecitabine) present in the nanoparticle composition is about 5% to about 95% by weight of the composition. In some embodiments, the drug load is about 10% to about 50%. In some embodiments, the drug load is about 20% to about 30%. Thus, in some embodiments, the drug load of active ingredient is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the nanoparticle composition has a molar ratio of at least one active ingredient (e.g., capecitabine) to lipid of about 90:10 to about 10:90. Thus, in some embodiments, the molar ratio of at least one active ingredient to lipid is about 90:10, about 80:20, about 70:30, about 50:50, about 60:40, about 40:60, about 30:70, about 20:80, or about 10:90. In some embodiments, the molar ratio of at least one active ingredient to lipid is about 50:50. In some embodiments, the molar ratio of at least one active ingredient to lipid is about 30:70.

In some embodiments, the nanoparticle composition comprises a phospholipids core comprising one or more lipids described herein and one or more active agents at least comprising capecitabine or its metabolites, and at least one layer of one or more polymers on the surface of the phospholipids core. In some embodiments, the one or more polymers comprise at least one of poly(lactic-co-glycolic acid) (PLGA) or its PEGylated form PEG-PLGA, polylactic acid (PLA) or its PEGylated form PEG-PLA, polyglycolic acid (PGA) or its PEGylated form PEG-PGA, poly-L-lactide-co-ε-caprolactone (PLCL) or its PEGylated form PEG-PLCL, Hyaluronic acid (HA) or its PEGylated form PEG-HA, poly(-L-lysine) (PLL) or its PEGylated form PEG-PLL, polyacrylic acid (PAA) or its PEGylated form PEG-PAA, polyphosphate (polyP), poly(acrylic acid-co-maleic acid), poly(butylene succinate), poly(alkyl cyanoacrylate) (PAC) or its PEGylated form PEG-PAC, or combinations thereof. In various embodiments, the nanoparticle composition may comprise 1,2-dipalmitoyl-sn-glycero-3-phosphocholine (DPPC), 1,2-dipalmitoyl-sn-glycero-3-phosphoethanolamine (DPPE-PEG), L-α-phosphatidylcholine (L-α-PC), 1,2-dilinoleoyl-sn-glycero-3-phosphocholine (DLPC), or 1,2-dioleoyl-3-trimethylammonium-propane (DOTAP) or combinations thereof. In some embodiments, the polymer comprises poly(lactic-co-glycolic acid) (PLGA). In some embodiments, the polymer comprises polyacrylic acid (PAA) or its PEGylated form PEG-PAA. In some embodiments, the polymer comprises polyphosphate (polyP).

In some embodiments, the nanoparticle composition has a molar ratio of lipid to polymer of about 100:0 to about 10:80. Thus, in some embodiments, the molar ratio of lipid to polymer is about 100:0, about 95:5, about 90:10, about 80:20, about 70:30, about 50:50, about 60:40, about 40:60, about 30:70, about 20:80, or about 10:90. In some embodiments, the molar ratio of lipid to polymer is about 90:10. In some embodiments, the molar ratio of lipid to polymer is about 70:30. In some embodiments, the molar ratio of lipid to polymer is about 50:50.

In some embodiments, the nanoparticle composition has a molar ratio of at least one active ingredient (e.g., capecitabine) to polymer of about 100:0 to about 10:90. Thus, in some embodiments, the molar ratio of at least one active ingredient to polymer is about 100:0, about 95:5, about 90:10, about 80:20, about 70:30, about 50:50, about 60:40, about 40:60, about 30:70, about 20:80, or about 10:90. In some embodiments, the molar ratio of at least one active ingredient to polymer is about 90:10. In some embodiments, the molar ratio of at least one active ingredient to polymer is about 70:30. In some embodiments, the molar ratio of at least one active ingredient to polymer is about 50:50.

In some embodiments, the nanoparticles further comprise at least one targeting agent, wherein the targeting agent selectively targets the nanoparticle to diseased tissue/cells, thereby minimizing whole body dose. In some embodiments, the targeting agent comprises an antibody or functional fragment thereof that is capable of recognizing a target antigen; and/or selected from an antibody, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, or an isotope or combinations thereof. The targeting agents may be attached by insertion of hetero/homo bifunctional spacer capable of reacting with amines of lipids and targeting moieties

In some embodiments, the nanoparticle composition has an average nanoparticle size of about 2.5 nm to about 200 nm. Thus, in some embodiments, the nanoparticle composition has an average nanoparticle size of about 2.5 nm, about 5 nm, about 10 nm, about 20 nm, about 30 nm, about 40 nm, about 50 nm, about 60 nm, about 70 nm, about 80 nm, about 90 nm, about 100 nm, about 120 nm, about 140 nm, about 160 nm, about 180 nm, or about 200 nm. In some embodiments, the nanoparticle composition has an average nanoparticle size of about 2.5 nm. In some embodiments, the nanoparticle composition has an average nanoparticle size of about 10 nm. In some embodiments, the nanoparticle composition has an average nanoparticle size of about 30 nm. In some embodiments, the nanoparticle composition has an average nanoparticle size of about 90 nm. In some embodiments, the nanoparticle composition has an average nanoparticle size of about 120 nm. In some embodiments, the nanoparticle composition has an average nanoparticle size of about 150 nm. In some embodiments, the nanoparticle composition has an average nanoparticle size of about 200 nm.

Certain embodiments can be described as intravenous and/or subcutaneous administration of a novel formulation of synthesized capecitabine bound to PLGA and a liposome. Such formulation is designed to offer a sustained release of capecitabine as active agent. Reference is made to the reduction of side effects due to the incorporation of a polymer and liposomal components of the formulation.

### Lipids for Use in the Nanoparticle Compositions

In some embodiments, the lipid used in the nanoparticle composition is an oil. In general, any oil known in the art can be conjugated to the polymers used in the disclosure. In some embodiments, an oil may comprise one or more fatty acid groups or salts thereof. In some embodiments, a fatty acid group may comprise digestible, long chain (e.g., Cg-Cso), substituted or unsubstituted hydrocarbons. In some embodiments, a fatty acid group may be a C10-C20 fatty acid or salt thereof. In some embodiments, a fatty acid group may be a C15-C20 fatty acid or salt thereof. In some embodiments, the fatty acid is saturated. In some embodiments, the fatty acid is unsaturated. In some embodiments, a fatty acid group is monounsaturated. In some embodiments, a fatty acid group is polyunsaturated. In some embodiments, a double bond of an unsaturated fatty acid group is in the cis conformation. In some embodiments, a double bond of an unsaturated fatty acid is in the trans conformation.

In some embodiments, a fatty acid group can be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid. Exemplary and non-limiting lipids are provided in Table 1.

TABLE 1
List of phospholipids and their properties at pH 7.

| | | Head group | Number of | Saturated/ |
|---|---|---|---|---|
| IUPAC nomenclature | Acronyms | charge | carbon tail | Unsaturated |
| 1,2-Didecanoyl-sn-glycero-3- | DDPC | Neutral | 10 | Unsaturated |
| phosphocholine | | | | |
| 1,2-dilauroyl-sn-glycero-3- | DLPE | Neutral | 12 | Unsaturated |
| phosphoethanolamine | | | | |
| 1,2-dimyristoyl-sn-glycero-3- | DMPC | Neutral | 14 | Saturated |
| phosphocholine | | | | |
| 1,2-dimyristoyl-sn-glycero-3- | DMPG | Negative | 14 | Saturated |
| phospho-(1′-rac-glycerol) | | | | |
| 1,2-dimyristoyl-sn-glycero-3- | DMPE- | Neutral | 14 | Saturated |
| phosphoethanolamine | PEG | | | |
| 1-palmitoyl-2-myristoyl-sn- | PMPC | Neutral | 14-16 | Saturated |
| glycero-3-phosphocholine | | | | |
| 1,2-dipalmitoyl-sn-glycero-3- | DPPG | Negative | 16 | Saturated |
| phospho-(1′-rac-glycerol) | | | | |
| 1,2-dipalmitoyl-sn-glycero-3- | DPPC* | Neutral | 16 | Saturated |
| phosphocholine | | | | |
| 1,2-dipalmitoyl-sn-glycero-3- | DPPE- | Neutral | 16 | Saturated |
| phosphoethanolamine | PEG* | | | |
| 1,2-dipalmitoyl-sn-glycero-3- | DPPA | Negative | 16 | Saturated |
| phosphate (sodium salt) | | | | |
| 1-palmitoyl-2-oleoyl-sn- | POPC | Neutral | 16-18 | Unsaturated |
| glycero-3-phosphocholine | | | | |
| 1-palmitoyl-2-stearoyl-sn- | PSPC | Neutral | 16-18 | Saturated |
| glycero-3-phosphocholine | | | | |
| 1-stearoyl-2-palmitoyl-sn- | SPPC | Neutral | 16-18 | |
| glycero-3-phosphocholine | | | | |
| 1,2-distearoyl-sn-glycero-3- | DEPE- | Neutral | 18 | Saturated |
| phosphoethanolamine-N- | PEG | | | |
| [methoxy(polyethylene glycol)- | | | | |
| 2000] | | | | |
| 1,2-distearoyl-sn-glycero-3- | DSPE- | Neutral | 18 | Saturated |
| phosphoethanolamine-N- | PEG | | | |
| [dibenzocyclooctyl(polyethylene | | | | |
| glycol)-2000] | | | | |
| L-α-phosphatidylcholine | L-α-PC* | Neutral | 18 | Mixture of |
| | | | | unsaturated |
| | | | | and saturated |
| 1,2-dilinoleoyl-sn-glycero-3- | DLPC* | Neutral | 18 | Unsaturated |
| phosphocholine | | | | |
| 1,2-dioleoyl-sn-glycero-3- | DOPG | Negative | 18 | Unsaturated |
| phospho-(1′-rac-glycerol) | | | | |
| 1,2-distearoyl-sn-glycero-3- | DSPG | Negative | 18 | Saturated |
| phospho-(1′-rac-glycerol) | | | | |
| 1,2-Distearoyl-3- | DSTAP | Positive | 18 | Saturated |
| trimethylammonium-propane | | | | |
| 1,2-dioleoyl-3- | DOTAP* | Positive | 18 | Unsaturated |
| trimethylammonium-propane | | | | |
| 1,2-dioleoyl-sn-glycero-3- | DOPA | Negative | 18 | Unsaturated |
| phosphate | | | | |
| 1,2-dioleoyl-sn-glycero-3- | DOPE | Neutral | 18 | Unsaturated |
| phosphoethanolamine | | | | |
| 1-stearoyl-2-oleoyl-sn-glycero- | SOPC | Neutral | 18 | Unsaturated |
| 3-phosphocholine | | | | |
| 1,2-dioleoyl-sn-glycero-3- | DOPC | Neutral | 18 | Unsaturated |
| phosphocholine | | | | |

*Refers to lipids specifically exemplified

### Polymers for Use in the Nanoparticle Compositions

A wide variety of polymers and methods for forming particles therefrom are known in the art of drug delivery. In some embodiments of the disclosure, the matrix of a particle comprises one or more polymers. Any polymer may be used in accordance with the present disclosure. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present disclosure are organic polymers.

A “polymer,” as used herein, is given its ordinary meaning as used in the art, i.e., a molecular structure comprising one or more repeat units (monomers), connected by covalent bonds. The repeat units of polymers suitable for the nanoparticle compositions described herein may all be identical, or in some cases, there may be more than one type of repeat unit present within the polymer. In some cases, the polymer is biologically derived, i.e., a biopolymer. Non-limiting examples of biopolymers include peptides or proteins (i.e., polymers of various amino acids), or nucleic acids such as DNA or RNA. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Various embodiments of the present disclosure are directed to copolymers, which, in particular embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer is a first block of the block copolymer and the second polymer is a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties). Alternatively, as described below, a copolymer can be formed using a lipid linker (e.g., DSPE).

In one set of embodiments, a polymer (e.g., copolymer, e.g., block copolymer) of the present disclosure includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the nanoparticles of the present disclosure can be “non-immunogenic.” The term “non-immunogenic” as used herein refers to endogenous growth factor in its native state, which normally elicits no, or only minimal levels of, circulating antibodies, T-cells, or reactive immune cells, and which normally does not elicit in the individual an immune response against itself.

It will be recognized that “biocompatibility” is a relative term, and some degree of immune response is to be expected even for polymers that are highly compatible with living tissue. However, as used herein, “biocompatibility” refers to the acute rejection of material by at least a portion of the immune system, i.e., a non biocompatible material implanted into a subject provokes an immune response in the subject that is severe enough such that the rejection of the material by the immune system cannot be adequately controlled, and often is of a degree such that the material must be removed from the subject. One simple test to determine biocompatibility is to expose a polymer to cells in vitro; biocompatible polymers are polymers that typically will not result in significant cell death at moderate concentrations, e.g., at concentrations of 50 micrograms/10 cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present disclosure include polydioxanone (PDO), polyhydroxyalkanoate, polyhydroxybutyrate, poly(glycerol sebacate), polyglycolide, polylactide, PLGA, polycaprolactone, or copolymers or derivatives including these and/or other polymers.

In certain embodiments, the biocompatible polymer is biodegradable, i.e., the polymer is able to degrade, chemically and/or biologically, within a physiological environment, such as within the body. For instance, the polymer may be one that hydrolyzes spontaneously upon exposure to water (e.g., within a subject), the polymer may degrade upon exposure to heat (e.g., at temperatures of about 370° C.). Degradation of a polymer may occur at varying rates, depending on the polymer or copolymer used. For example, the half-life of the polymer (the time at which 50% of the polymer is degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

Further suitable polymers include polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” Exemplary polyesters suitable for the embodiments described herein include, for example, polyhydroxyacids; PEGylated polymers and copolymers of lactide and glycolide (e.g., PEGylated PLA, PEGylated PGA, PEGylated PLGA, and derivatives thereof). Additional polyesters include, for example, polyanhydrides, poly(ortho ester) PEGylated poly(ortho ester), poly(caprolactone), PEGylated poly(caprolactone), polylysine, PEGylated polylysine, poly(ethylene inline), PEGylated poly(ethylene imine), poly(L-lactide-co-L-lysine), poly(serine ester), poly(4-hydroxy-L-proline ester), poly[a-(4-aminobutyl)-L-glycolic acid], and derivatives thereof.

In some embodiments, the polymer is PLGA. PLGA is a biocompatible and biodegradable co-polymer of lactic acid and glycolic acid, and various forms of PLGA are characterized by the ratio of lactic acid: glycolic acid. Lactic acid can be in the form of L-lactic acid, D-lactic acid, or D,L-lactic acid. The degradation rate of PLGA can be adjusted by altering the lactic acid-glycolic acid ratio. In some embodiments, PLGA is characterized by a lactic acid: glycolic acid ratio of approximately 85:15, approximately 75:25, approximately 60:40, approximately 50:50, approximately 40:60, approximately 25:75, or approximately 15:85.

In particular embodiments, by optimizing the ratio of lactic acid to glycolic acid monomers in the polymer of the nanoparticle (e.g., the PLGA block copolymer or PLGA-PEG block copolymer), nanoparticle parameters such as water uptake, therapeutic agent release (e.g., “controlled release”) and polymer degradation kinetics can be optimized. In some embodiments, the polymer is an acrylic polymers. Suitable and non-limiting acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, the polymer is a cationic polymers. In general, cationic polymers are able to condense and/or protect negatively charged strands of nucleic acids (e.g. DNA, RNA, or derivatives thereof). Amine-containing polymers such as poly(lysine) (Zauner et ah, 1998, Adv. Drug Del. Rev., 30:97; and Kabanov et ah, 1995, Bioconjugate Chem., 6:7), polyethylene imine) (PEI; Boussif et al, 1995, Proc. Natl. Acad. ScL, USA, 1995, 92:7297), and poly(amidoamine) dendrimers (Kukowska-Latallo et al, 1996, Proc. Natl. Acad. ScL, USA, 93:4897; Tang et al, 1996, Bioconjugate Chem., 7:703; and Haensler et al, 1993, Bioconjugate Chem., 4:372) are positively charged at physiological pH, form ion pairs with nucleic acids, and mediate transfection in a variety of cell lines.

In some embodiments, the polymer is a degradable polyesters bearing cationic side chains (Putnam et al, 1999, Macromolecules, 32:3658; Barrera et al., 1993,/. Am. Chem. Soc, 115:11010; Kwonef al, 19% 9, Macromolecules, 22325Q-, Urn et al., 1999, J. Am. Chem. Soc, 121:5633; and Zhou et al, 1990, Macromolecules, 23:3399). Examples of these polyesters include poly(L-lactide-co-L-lysine) (Barrera et al, 1993,/. Am. Chem. Soc, 115:11010), poly(serine ester) (Zhou et al, 1990, Macromolecules, 23:3399), poly(4-hydroxy-L-proline ester) (Putnam et ah, 1999, Macromolecules, 32:3658; and Lim et al, 1999,/. Am. Chem. Soc, 121:5633). Poly(4-hydroxy-L-proline ester) was demonstrated to condense plasmid DNA through electrostatic interactions, and to mediate gene transfer (Putnam et al, 1999, Macromolecules, 32:3658; and Lim et al, 1999,/. Am. Chem. Soc, 121:5633). These new polymers are less toxic than poly(lysine) and PEI, and they degrade into non-toxic metabolites. A polymer (e.g., copolymer, e.g., block copolymer) containing poly(ethylene glycol) repeat units is also referred to as a “PEGylated” polymer. Such polymers can control inflammation and/or immunogenicity (i.e., the ability to provoke an immune response) and/or lower the rate of clearance from the circulatory system via the reticuloendothelial system, due to the presence of the poly(ethylene glycol) groups. PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

In addition, certain embodiments of the disclosure are directed towards copolymers containing poly(ester-ether)s, e.g., polymers having repeat units joined by ester bonds (e.g., R—C(0)-0-R′ bonds) and ether bonds (e.g., R—O—R′ bonds). In some embodiments of the disclosure, a biodegradable polymer, such as a hydrolyzable polymer, containing carboxylic acid groups, is conjugated with poly(ethylene glycol) repeat units to form a poly(ester-ether).

In a particular embodiment, the molecular weight of the polymers of the nanoparticles of the disclosure is optimized for effective treatment of diseases, e.g., cancer. For example, the molecular weight of the polymer influences nanoparticle degradation rate (particularly when the molecular weight of a biodegradable polymer is adjusted), solubility, water uptake, and drug release kinetics (e.g. “controlled release”). As a further example, the molecular weight of the polymer can be adjusted such that the nanoparticle biodegrades in the subject being treated within a reasonable period of time (ranging from a few hours to 1-2 weeks, 3-4 weeks, 5-6 weeks, 7-8 weeks, etc.).

In particular embodiments is a nanoparticle composition comprising a copolymer of PEG and PLGA. In one aspect, the PEG has a molecular weight of about 1,000-20,000 Da, about 5,000-20,000 Da, or about 10,000-20,000 Da, and the PLGA has a molecular weight of about 5,000-100,000 Da, about 20,000-70,000 Da or about 20,000-50,000 Da.

In certain embodiments, the polymers of the nanoparticles may be conjugated to a lipid, i.e., a lipid in addition to the amphiphilic component of the nanoparticle of the disclosure. The polymer may be, for example, a lipid-terminated PEG. The present disclosure also provides methods for forming amphiphilic-protected nanoparticles with a lipid-terminated PEG. For example, such a method comprises providing a first polymer that is reacted with a lipid, to form a polymer/lipid conjugate. The polymer/lipid conjugate is then reacted with a targeting moiety to prepare a targeting moiety-bound polymer/lipid conjugate; and mixing the ligand-bound polymer/lipid conjugate with a second, non-functionalized polymer, an amphiphilic component, and a therapeutic agent; such that an amphiphilic layer-protected nanoparticle is formed. In certain embodiments, the first polymer is PEG, such that a lipid-terminated PEG is formed. The lipid terminated PEG can then, for example, be mixed with PLGA to form a nanoparticle. As described above, the lipid portion of the polymer can be used for self-assembly with another polymer, facilitating the formation of a nanoparticle. For example, a hydrophilic polymer could be conjugated to a lipid that will self-assemble with a hydrophobic polymer.

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al, 2001. Am. Chem. Soc, 123:9480; Lim et al., 2001. Am. Chem. Soc, 123:2460; Langer, 2000. Ace. Chem. Res., 33:94; Langer, 1999. Control. Release, 62:7; and Uhrich et al, 1999. Chem. Rev., 99:3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons)

### Methods of Manufacturing Nanoparticle Compositions of Capecitabine

Some embodiments described herein are methods for forming a nanoparticle composition comprising forming an organic phase by combining one or more phospholipids, one or more polymers, one or more solvents and at least one of capecitabine or its metabolites; forming a lipid aqueous phase by combining one or more targeting agents with water; mixing the organic phase with the aqueous phase in a multi-inlet vortex mixer, whereby self-assembly of micelles occurs; spray drying or freeze drying the suspension and recycling the organic solvents; and mixing solution with one or more polymers with the micelles, whereby layer-by-layer polymer deposition occurs and wherein the capecitabine or its metabolites nanoparticles provide sustained release of active components when provided to a subject. In some embodiments, the solvent is selected from ethanol, methanol, tetrahydrofuran, acetonitrile, acetone, tert butyl alcohol, dimethyl formamide, and hexafluro isopropanol. In some embodiments, the one or more active agents comprise capecitabine or its metabolites; and/or at least one anti-cancer drug; and/or a radioisotope; and/or at least one active agent selected from fluorescent dyes, quantum dots, iron, silver, gold, and platinum, and combinations thereof.

In certain aspects, mixing the organic phase with the lipid aqueous phase comprises vigorous micromixing in the multi-inlet vortex mixer; and/or mixing the organic phase with the lipid aqueous phase comprises vortexing; and/or mixing the organic phase with the lipid aqueous phase further comprises sonicating. In certain aspects, the method further comprises organic solvent removal; and/or dialysis; and/or freezing the nanoparticles; and/or lyophilizing the nanoparticles; and/or spray-drying the particles; and/or attaching a targeting agent to the nanoparticles; and/or attaching at least one targeting agent, wherein the targeting agent selectively targets the nanoparticle to diseased tissue/cells, thereby minimizing whole body dose; and/or attaching at least one targeting agent to the nanoparticles, wherein the targeting agent comprises an antibody or functional fragment thereof that is capable of recognizing a target antigen.

In another embodiment, the disclosure provides an amphiphilic layer-protected nanoparticle, and methods of making the nanoparticle, wherein one polymer of the polymeric matrix (e.g., PEG), is conjugated to a lipid that will self-assemble with another polymer (e.g., PLGA), such that the polymers of the polymeric matrix are not covalently bound, but are bound through self-assembly. “Self-assembly” refers to a process of spontaneous assembly of a higher order structure that relies on the natural attraction of the components of the higher order structure (e.g., molecules) for each other. Self-assembly typically occurs through random movements of the molecules and formation of bonds based on size, shape, composition, or chemical properties. The lipid that is used for self-assembly of the polymers is in addition to the amphiphilic component of the nanoparticle.

### Pharmaceutical Compositions

Certain embodiments described herein are pharmaceutical compositions comprising the nanoparticle composition described herein and a pharmaceutically acceptable carrier. In some embodiments, the pharmaceutical composition is formulated for the intravenous and/or subcutaneous administration of a novel formulation of synthesized capecitabine in a nanoparticle composition described herein. As used herein, the term “pharmaceutically acceptable carrier” means a nontoxic, inert solid, semi-solid or liquid filler, diluent, encapsulating material or formulation auxiliary of any type. Remington's Pharmaceutical Sciences. Ed. By Gennaro, Mack Publishing, Easton, Pa., 1995 discloses various carriers used in formulating pharmaceutical compositions and known techniques for the preparation thereof. Some exemplary and non-limiting materials which can serve as pharmaceutically acceptable carriers include, but are not limited to, sugars such as lactose, glucose, and sucrose; starches such as com starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN® 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. If filtration or other terminal sterilization methods are not feasible, the formulations can be manufactured under aseptic conditions.

The pharmaceutical compositions of this disclosure can be administered to a patient by any means known in the art including oral and parenteral routes. In certain embodiments, parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays). In a particular embodiment, the nanoparticles of the present disclosure are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are employed as a solvent or suspending medium. For this purpose, any bland fixed oil can be employed including synthetic mono- or diglycerides. In addition, fatty acids such as oleic acid are used in the preparation of injectables. In one embodiment, the inventive nanoparticle composition is suspended in a carrier fluid comprising 1% (w/v) sodium carboxymethyl cellulose and 0.1% (v/v) TWEEN® 80. The injectable formulations can be sterilized, for example, by filtration through a bacteria-retaining filter, or by incorporating sterilizing agents in the form of sterile solid compositions, which can be dissolved or dispersed in sterile water or other sterile injectable medium prior to use.

Compositions for rectal or vaginal administration may be suppositories, which can be prepared by mixing the inventive nanoparticle composition with suitable non-irritating excipients, or carriers such as cocoa butter, polyethylene glycol, or a suppository wax which are solid at ambient temperature but liquid at body temperature and therefore melt in the rectum or vaginal cavity and release the inventive nanoparticle composition. Dosage forms for topical or transdermal administration of an inventive pharmaceutical composition include ointments, pastes, creams, lotions, gels, powders, solutions, sprays, inhalants, or patches. The inventive nanoparticle composition is admixed under sterile conditions with a pharmaceutically acceptable carrier and any needed preservatives or buffers as may be required. Ophthalmic formulations, eardrops, and eye drops are also contemplated as being within the scope of this disclosure. The ointments, pastes, creams, and gels may contain, in addition to the inventive nanoparticle compositions of this disclosure, excipients such as animal and vegetable fats, oils, waxes, paraffins, starch, tragacanth, cellulose derivatives, polyethylene glycols, silicones, bentonites, silicic acid, talc, and zinc oxide, or mixtures thereof. Transdermal patches have the added advantage of providing controlled delivery of a compound to the body. Such dosage forms can be made by dissolving or dispensing the inventive nanoparticle compositions in a proper medium. Absorption enhancers can also be used to increase the flux of the compound across the skin. The rate can be controlled by either providing a rate controlling membrane or by dispersing the inventive nanoparticle compositions in a polymer matrix or gel.

Powders and sprays can contain, in addition to the inventive nanoparticle compositions of this disclosure, excipients such as lactose, talc, silicic acid, aluminum hydroxide, calcium silicates, and polyamide powder, or mixtures thereof. Sprays can additionally contain customary propellants such as chlorofluorohydrocarbons. When administered orally, the inventive nanoparticles can be, but are not necessarily, encapsulated. A variety of suitable encapsulation systems are known in the art (“Microcapsules and Nanoparticles in Medicine and Pharmacy,” Edited by Doubrow, M., CRC Press, Boca Raton, 1992; Mathiowitz and Langer J. Control. Release 5:13, 1987; Mathiowitz et al. Reactive Polymers 6:275, 1987; Mathiowitz et al. J. Appl. Polymer Sci. 35:755, 1988; Langer Ace. Chem. Res. 33:94, 2000; Langer J. Control. Release 62:7, 1999; Uhrich et al. Chem. Rev. 99:3181, 1999; Zhou et al. J. Control. Release 75:27, 2001; and Hanes et al. Pharm. Biotechnol. 6:389, 1995). The inventive nanoparticle compositions may be encapsulated within biodegradable polymeric microspheres or liposomes. Examples of natural and synthetic polymers useful in the preparation of biodegradable microspheres include carbohydrates such as alginate, cellulose, polyhydroxyalkanoates, polyamides, polyphosphazenes, polypropylfumarates, polyethers, polyacetals, polycyanoacrylates, biodegradable polyurethanes, polycarbonates, polyanhydrides, polyhydroxyacids, poly(ortho esters), and other biodegradable polyesters. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Pharmaceutical compositions for oral administration can be liquid or solid. Liquid dosage forms suitable for oral administration of inventive compositions include pharmaceutically acceptable emulsions, microemulsions, solutions, suspensions, syrups, and elixirs. In addition to an encapsulated or unencapsulated nanoparticle composition, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants, wetting agents, emulsifying and suspending agents, sweetening, flavoring, and perfuming agents. As used herein, the term “adjuvant” refers to any compound, which is a nonspecific modulator of the immune response. In certain embodiments, the adjuvant stimulates the immune response. Any adjuvant may be used in accordance with the present disclosure. A large number of adjuvant compounds is known in the art (Allison Dev. Biol. Stand. 92:3-11, 1998; Unkeless et al. Annu. Rev. Immunol. 6:251-281, 1998; and Phillips et al. Vaccine 10:151-158, 1992).

Solid dosage forms for oral administration include capsules, tablets, pills, powders, and granules. In such solid dosage forms, the encapsulated or unencapsulated nanoparticle composition is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

It will be appreciated that the exact dosage of the pharmaceutical composition is chosen by the individual physician in view of the patient to be treated, in general, dosage and administration are adjusted to provide an effective amount of the pharmaceutical composition to the patient being treated.

The nanoparticles of the disclosure may be formulated in dosage unit form for ease of administration and uniformity of dosage. The expression “dosage unit form” as used herein refers to a physically discrete unit of nanoparticle appropriate for the patient to be treated. It will be understood, however, that the total daily usage of the compositions of the present disclosure will be decided by the attending physician within the scope of sound medical judgment. For any nanoparticle, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions, which exhibit large therapeutic indices, may be useful in some embodiments. The data obtained from cell culture assays and animal studies can be used in formulating a range of dosages for human use.

The present disclosure also provides any of the above-mentioned compositions in kits, optionally with instructions for administering any of the compositions described herein by any suitable technique as previously described, for example, orally, intravenously, pump or implantable delivery device, or via another known route of drug delivery. “Instructions” can define a component of promotion, and typically involve written instructions on or associated with packaging of compositions of the disclosure. Instructions also can include any oral or electronic instructions provided in any manner. The “kit” typically defines a package including any one or a combination of the compositions of the disclosure and the instructions, but can also include the composition of the disclosure and instructions of any form that are provided in connection with the composition in a manner such that a clinical professional will clearly recognize that the instructions are to be associated with the specific composition. The kits described herein may also contain one or more containers, which may contain the inventive composition and other ingredients as previously described. The kits also may contain instructions for mixing, diluting, and/or administrating the compositions of the disclosure in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the nanoparticle and the mode of use or administration. Suitable solvents for drug compositions are well known, for example as previously described, and are available in the literature. The solvent will depend on the nanoparticle and the mode of use or administration.

### Methods

The disclosure also involves, in another aspect, promotion of the administration of any of the nanoparticles described herein. Some embodiments described herein are methods for treating a subject having a disease (e.g., cancer) including identifying a subject suspected of having a disease, and administering an effective amount of the nanoparticle composition or pharmaceutical composition comprising it in an effective amount to treat the disease. In some aspects, the administration of the composition reduces one or more of the side effects comprising nausea, vomiting, dermatitis, bone-marrow depression, cardiotoxicity or diarrhea or a combination thereof when provided to a subject compared to administration of capecitabine that is not formulated in the nanoparticle composition described herein.

In some embodiments, one or more compositions of the disclosure are promoted for the prevention or treatment of various diseases such as those described herein via administration of any one of the compositions of the present disclosure. As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the disclosure.

In further embodiments described herein, the compositions described herein may be used for the treatment of neoplastic diseases (cancer), and neurologic-auto-immunological degenerative diseases (Parkinson's disease, Alzheimer's disease, multiple sclerosis, ALS, sequel, behavioral and cognitive disorders, autism spectrum, and depression). In certain embodiments, the compositions of the present disclosure are administered intramuscular, subcutaneous and or intravascular.

The nanoparticle composition or pharmaceutical compositions described herein can be administered, for example, to a subject, or a subject in need thereof In one aspect, the subject is a mammal, or a mammal in need thereof. In one aspect, the subject is a human, or human in need thereof. In one aspect, the subject is a human. In one aspect, the subject is a child (˜0-9 years old) or an adolescent (˜10-17 years old). In one aspect, the subject is from about 0 to about 9 years of age. In another aspect, the subject is from about 10 years to about 17 years of age. In another aspect, the subject is over 17 years of age. In another aspect, the subject is an adult (≥18 years of age).

Some additional embodiments are a method of manufacturing a medicament comprising the nanoparticle composition of capecitabine for the treatment of one or more diseases described herein. The medicament can subsequently be administered to a patient in need thereof.

The following examples are intended to illustrate certain embodiments of the present disclosure, but do not exemplify the full scope of the disclosure.

### EXAMPLES

The disclosure is further illustrated by the following examples. These examples should not be construed as limiting.

### Example 1: Formation of CAP Nanoparticles

Structure of the Polymer-Lipid Nanoparticles of CAP

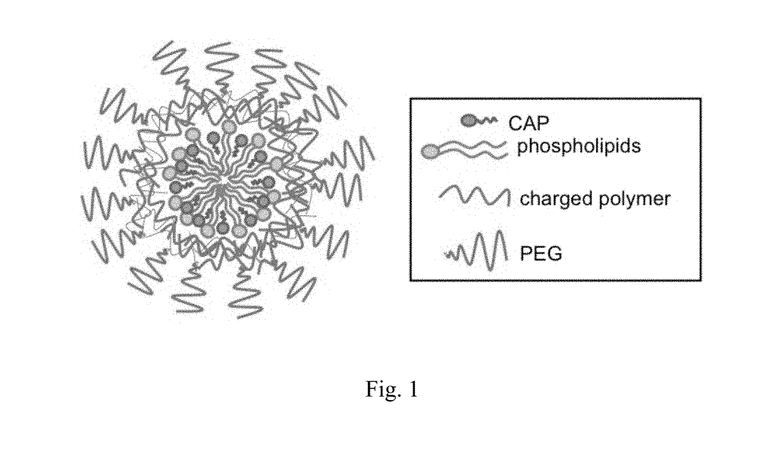

Capecitabine and phospholipids form micelle structures (see FIG. 1). Polymers of opposite charges were used to wrap the micelles by electrostatic interactions. If necessary, multiple layers of polymers of alternative charges could be deposited on the particles. Eventually, the outermost layer of polyethylene glycol (PEG) provides steric stability and long-blood circulation time.

Particle Formation Process

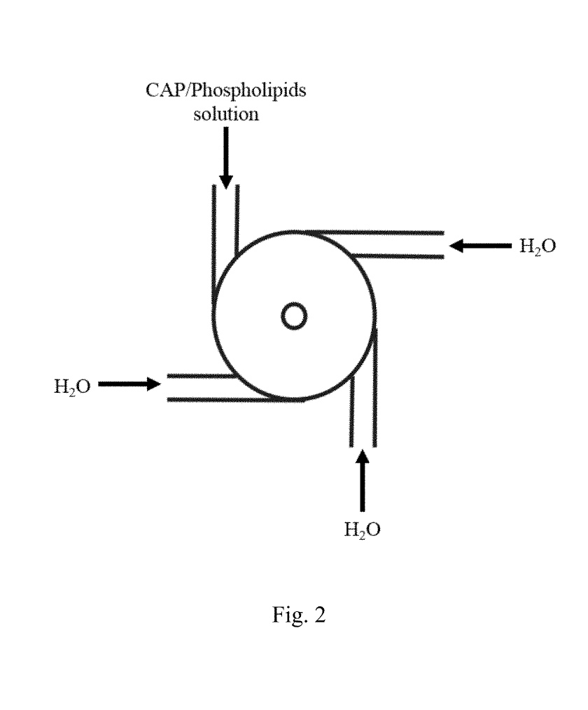

Lipid-capecitabine micelles were generated first through fast solvent exchange methods utilizing a multi-inlet vortex mixer (MIVM) followed by spray or freeze-drying together with leucine and trehalose. Specifically, MIVM was setup as illustrated in FIG. 2. The micelle suspension was freeze-dried for 48 hours. The dried powders were re-suspended in an aqueous solution by vigorous mixing or sonication. Polymers of opposite charges were added to the lipid-CAP micelles. PEGylated diblock copolymers were used at the end if multiple layers were deposited on the micelles to provide steric stability. The process employed the amphiphilic nature of capecitabine and controlled micromixing.

Amphiphilic Properties of Capecitabine

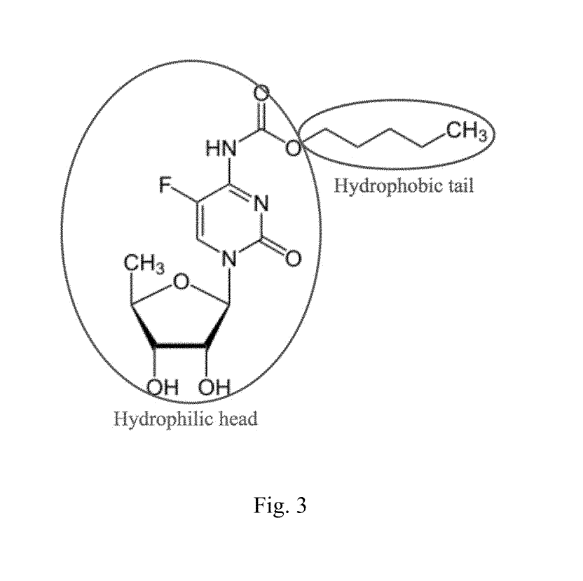

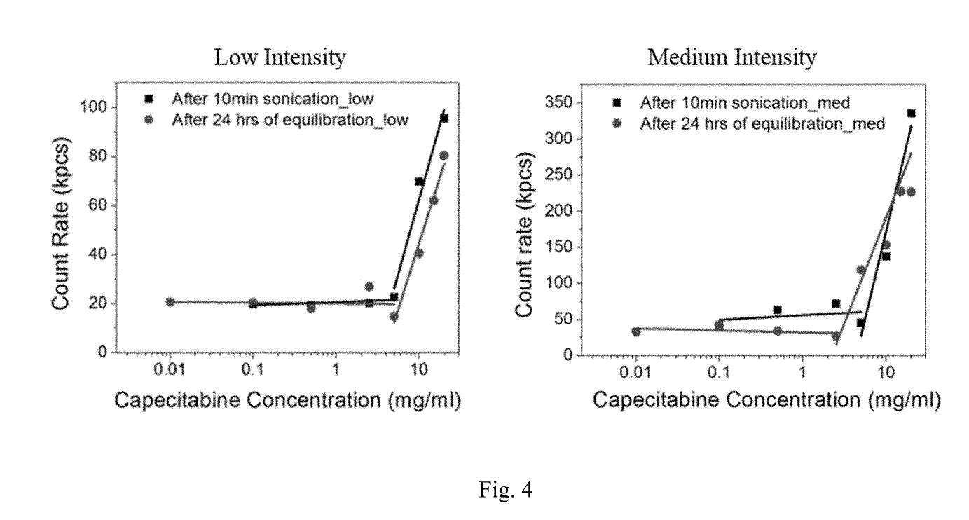

The molecule of CAP exhibits amphiphilic properties. Capecitabine molecule has a short carbon tail and a hydrophilic head group (contains hydroxyl groups) as shown in FIG. 3. Dynamic light scattering (DLS) was used to determine the solubility of CAP in deionized (DI) water. CAP concentration were varied between 0.01 mg/ml to 20 mg/ml. The count rate remains relatively low until CAP concentration reaches a few mg/ml. Sharp increase of the count rate indicates particle formation in the solution. The experiments were repeated at low laser intensity and medium intensity (FIG. 4). There was no significant difference in the results. Furthermore, all CAP solutions were left over night at room temperature and the measurements were repeated after. The results reproducibly show the sharp increase of scattering light intensity at about a few mg/ml concentration of CAP.

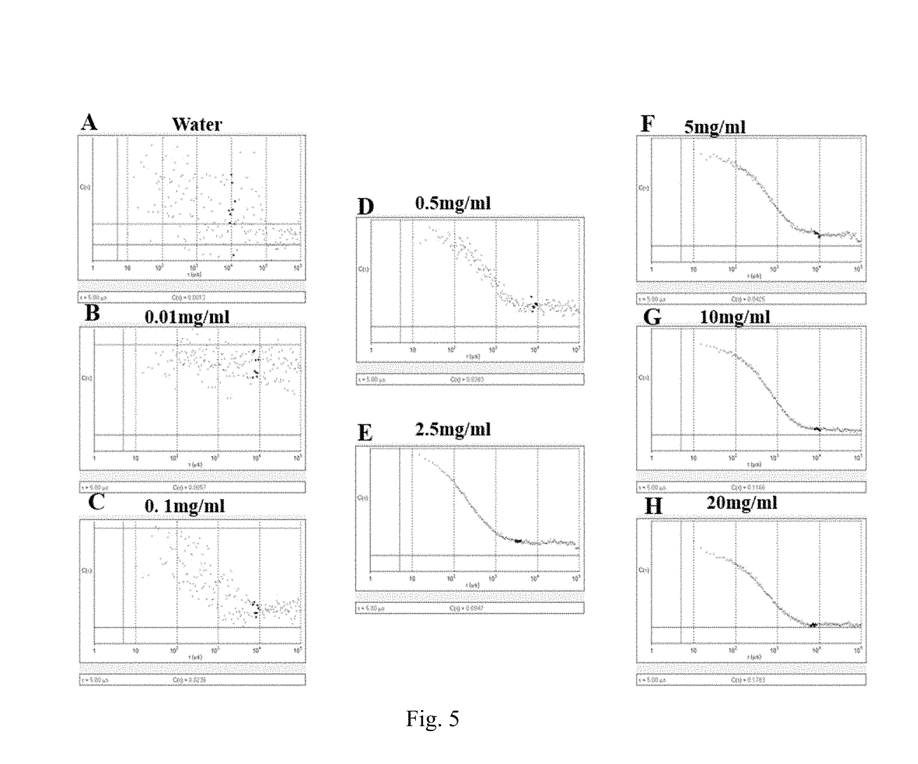

DLS was also used to decide the size of the particles, which is about 2-30 nm. Very likely, micelle structures were formed in the solutions. However, the size and correlation function fluctuates greatly at high concentrations of CAP, which indicated that CAP micelles were not very stable and equilibrium was fast (FIG. 5).

Lipid-CAP Micelles

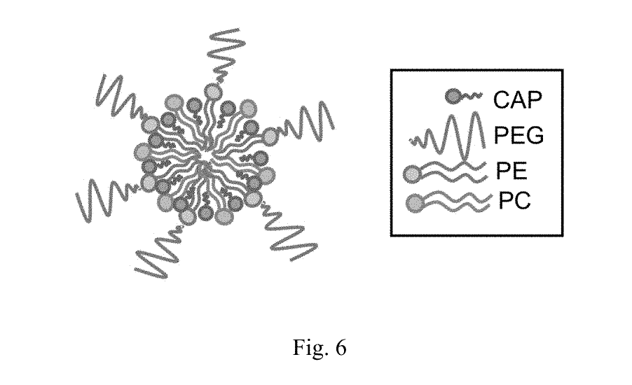

Lipids were added to CAP to control the properties of the micelles. A non-limiting and exemplary list of appropriate lipids is presented in Table 1 above. The structure of lipid-CAP micelles is presented in FIG. 6.

### Example 2: Formation of Lipid-CAP Micelles with Neutral Surfaces

Two processes have been used to generate the micelles—film hydration and continuous mixing. By film hydration, 215.6 μL of CAP (5 mg/ml) was added with 117.4 μL of DPPC (1,2-dipalmitoyl-sn-glycero-3-phosphocholine) (25 mg/ml) and 329.9 μL of DPPE-PEG (1,2-distearoyl-sn-glycero-3-phosphoethanolamine-N-[methoxy(polyethylene glycol)-2000] (25 mg/ml). The mixture is thoroughly mixed and evaporated under Argon stream and was placed in vacuum for at least 2 hours. Then, the mixture was rehydrated with water and sonicated for 10 minutes. By continuous mixing, CAP, DPPC and PEGylated lipid DPPE-PEG were dissolved in ethanol and quickly mixed with DI water in the MIVM at different volumes depending on the molar ratio. A typical condition is described as follows. CAP (2 mM), DPPC (2.67 mM) and DPPE-PEG (2 mM) were dissolved in ethanol CAP. The solution was loaded into 5 mL gastight syringe and quickly mixed with the other three streams, which were DI water. The stream of CAP/phospholipids solution and one of the water streams were pumped at 6 ml/min. The other two water streams were pumped at 54 ml/min. The micelle suspension was freeze-dried for 48 hours and resuspended in an aqueous solution. Molar ratios of CAP, DPPC, and DPPE-PEG are shown in Table 2.

TABLE 2
Information of micelles of CAP, DPPC and DPPE-PEG

| Molar Ratio | | | Drug loading | Average diameter |
|---|---|---|---|---|
| CAP | DPPC | DPPE-PEG | (wt %) | (nm) |
| 30 | 40 | 30 | 17.7 | 8.52 |
| 50 | 0 | 50 | 34.18 | 9.12 |

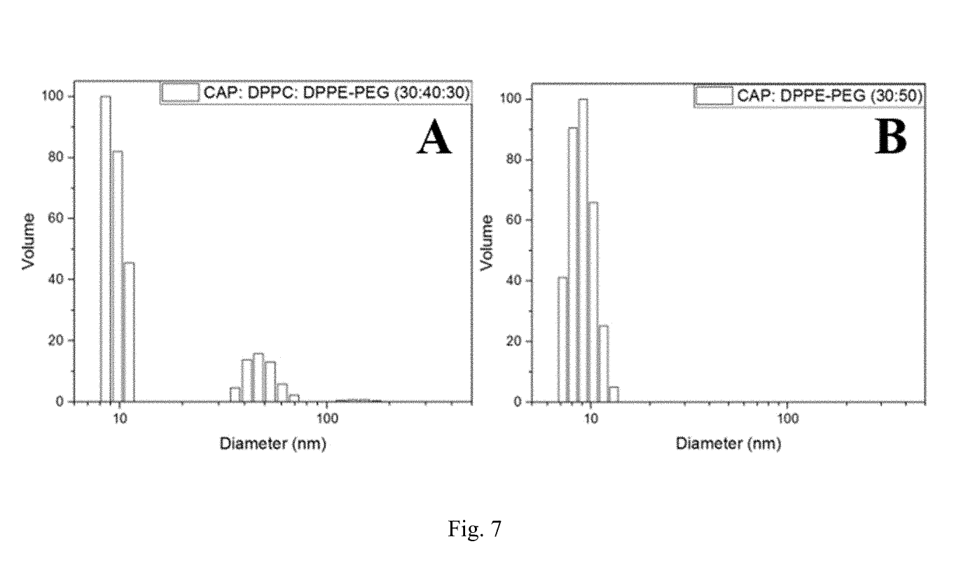

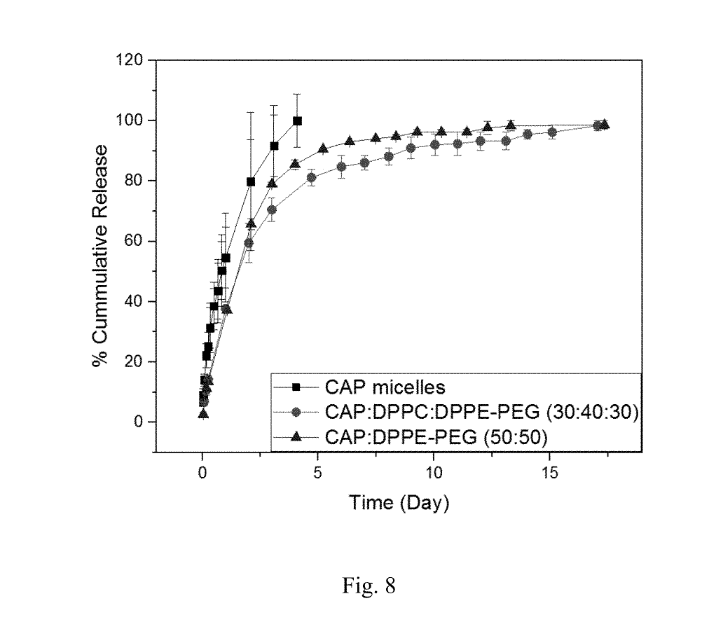

The sizes of these micelles were measured using DLS (FIG. 7). The average sizes of the micelles were reported in Table 2. The release of CAP from micelles were measured using the dialysis method. The micelles hinder the release of CAP to several days (FIG. 8).

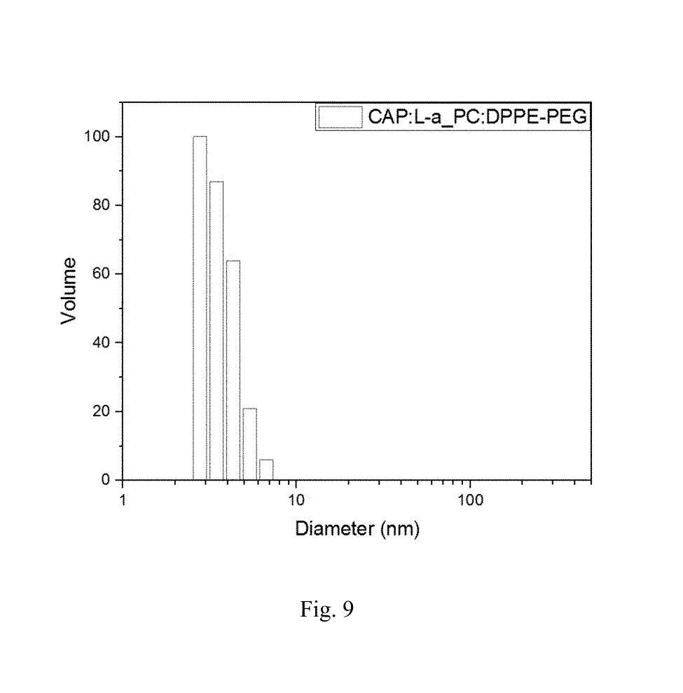

Other phospholipids were also utilized to formulate lipid micelles. Another examples of lipid micelles tested is the combination of L-α-phosphatidylcholine (L-α-PC), DPPC-PEG and CAP in the following molar ratio. Size distribution is shown in FIG. 9.

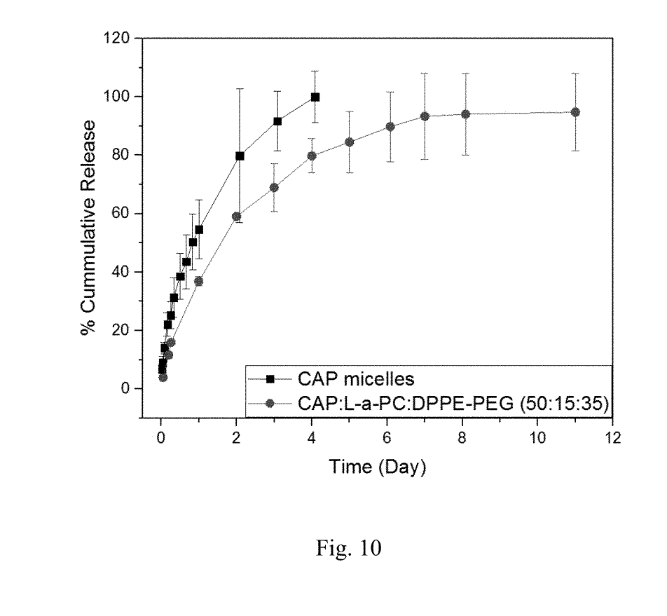

The release results showed that the release is slowed down when compared to the release of pure CAP (FIG. 10).

TABLE 3
Information of micelles of CAP, DPPC and DPPE-PEG

| Molar ratio | | | Theoretical drug | Average diameter |
|---|---|---|---|---|
| CAP | L-α-PC | DPPE-PEG | loading (wt %) | (nm) |
| 50 | 15 | 35 | 9.24 | 2.79 |

### Example 3: Formation of Lipid-CAP Micelles with Positively Charged Surfaces

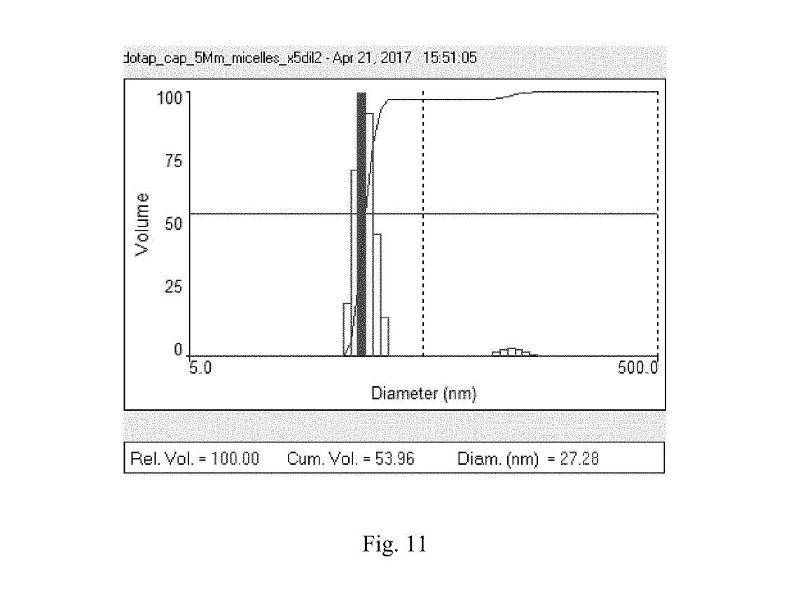

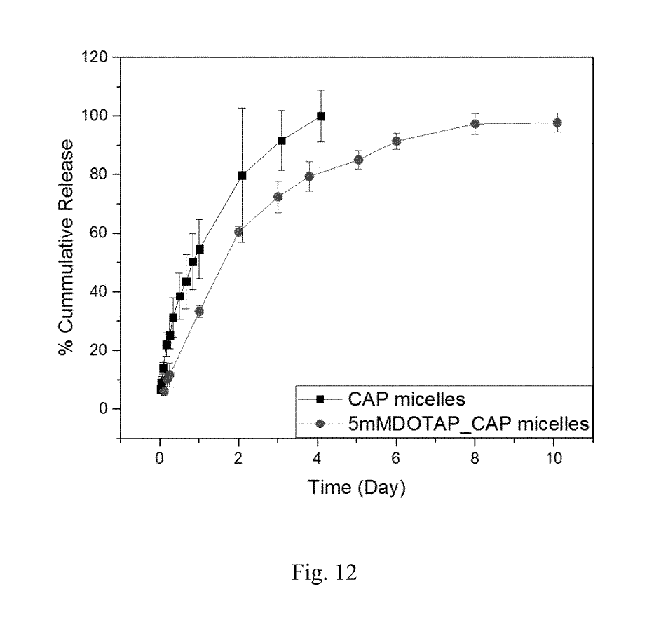

Two processes have been used to generate the micelles—film rehydration and continuous mixing. By film hydration, DOTAP (1,2-dioleoyl-3-trimethylammonium-propane) and CAP were both dissolved in chloroform at 10 mM concentration. Equal volume of DOTAP and CAP (500 ul) solutions were mixed together and the mixture was evaporated under Argon stream and then placed under vacuum for at least 2 hours to ensure complete evaporation of the solvent. The dried film was then rehydrated with 1 ml of DI water and sonicated for 10 minutes. By continuous mixing, cationic phospholipids such as DOTAP were used to make micelles with CAP. CAP (2 mM) and DOTAP (2 mM) were dissolved in ethanol. The solution was loaded into 5 mL gastight syringe and quickly mixed with the other three streams, which were DI water in the MIVM. The stream of CAP/phospholipids solution and one of the water streams were pumped at 6 ml/min. The other two water streams were pumped at 54 ml/min. The micelle suspension was freeze-dried for 48 hours and resuspended in an aqueous solution. Size distribution of the micelles is shown in FIG. 11. Release of CAP from DOTAP-CAP micelles is shown in FIG. 12.

TABLE 4
Information of DOTAP-CAP micelles.

| Concentration | | Theoretical drug | Average diameter | Average zeta |
|---|---|---|---|---|
| CAP | DOTAP | loading (wt %) | (nm) | potential |
| 5 mM | 5 mM | 33.97 | 30 nm | 55 mV |

Polymer-Lipid Hybrid Particles

Lipid-CAP micelles were further wrapped in polymers by a layer-by-layer deposition process to vary particle surface properties, achieve better stability, control particle size, and further sustain the release. This fully enable the control of the size of particles from a few nanometers to hundreds of nanometers and the surface charges designed to be negative, positive, or neutral. A list of FDA approved biocompatible and biodegradation polymers that are used for the formulation are listed in Table 5.

TABLE 5
List of anionic and cationic biodegradable polymers

| Anionic biopolymer | Cationic biopolymer |
|---|---|
| Alginate | Chitosan |
| Polyphosphate | ε-Polylysine |
| Pectin (at mildly acidic solution) | Dextran |
| Carrageenan | Poly(amide ester)s |
| Hyaluronic acid (HA) | poly(-L-lysine) (PLL) |
| Polyacrylic acid | |
| poly(lactic-co-glycolic acid (PLGA), | |
| acid terminated | |
| Polylactic acid (PLA) | |
| Polyglycolic acid (PGA) | |
| Poly-L-lactide-co-ε-caprolactone (PLCL) | |
| Poly(acrylic acid-co-maleic acid) | |
| Poly(butylene succinate) | |
| poly(alkyl cyanoacrylate) (PAC) | |

### Example 4: PolyP-DOTAP-CAP Nanoparticles

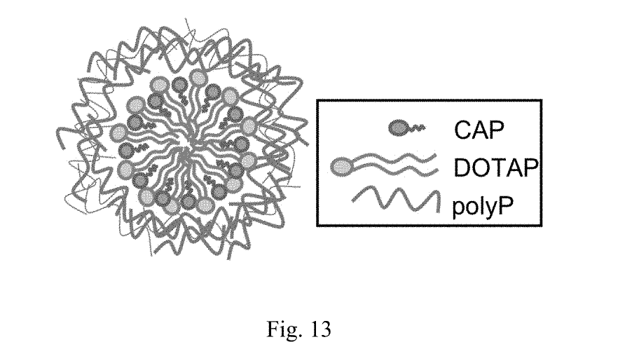

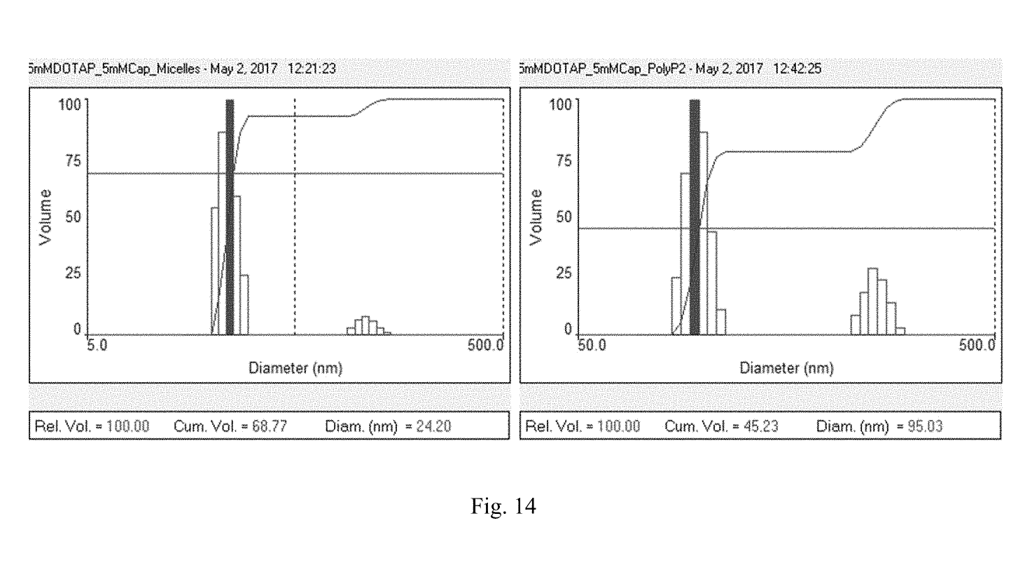

DOTAP-CAP micelles were first formulated as described in the Example 2. Polyphosphate (polyP, with 75 repeating units in average) was dissolved in water at 5 mM concentration. Resuspended DOTAP-CAP micelles was mixed with polyP solution at various ratios. The structure of polyO-DOTAP-CAP nanoparticles is shown in FIG. 13. Typical DLS measurements of particle size before and after polyP deposition are shown in FIG. 14.

TABLE 6
Information of the polyP-DOTAP-CAP nanoparticles.

| | | | | Average | Zeta |
|---|---|---|---|---|---|
| Concentrations | | | Theoretical drug | diameter | potential |
| DOTAP | CAP | PolyP | loading (wt %) | (nm) | (mV) |
| 2.5 mM | 2.5 mM | 2.5 mM | 23.85 | 127.4 | −45.7 |
| 2.5 mM | 2.5 mM | 1.25 mM | 28.02 | 119.3 | −51.8 |
| 2.5 mM | 2.5 mM | 0.625 mM | 30.71 | 152.4 | −54.8 |

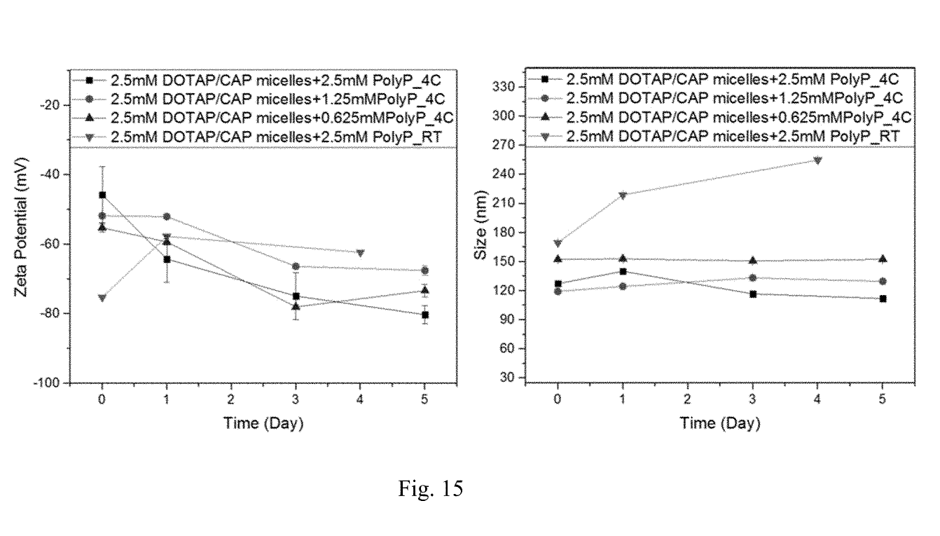

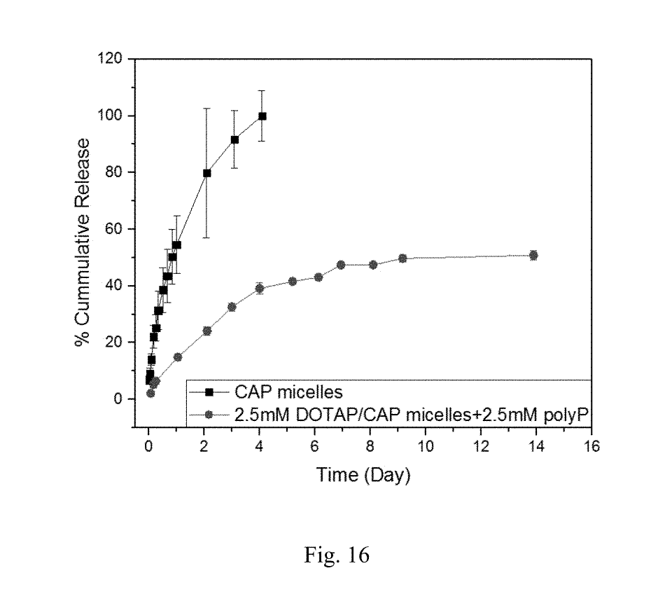

Stability of the polyP-DOTAP-CAP nanoparticles were monitored for five days at 4° C. and room temperature (FIG. 15). Zeta potential for all three samples decreases slightly over the course of 5 days. Size of the particles remained constant for five days at 4° C. and increased as a power-law function at room temperature. The release of CAP from these polyP-DOTAP-CAP nanoparticles was significantly sustained (FIG. 16).

### Example 5: PEG-PAA-DOTAP-CAP Nanoparticles

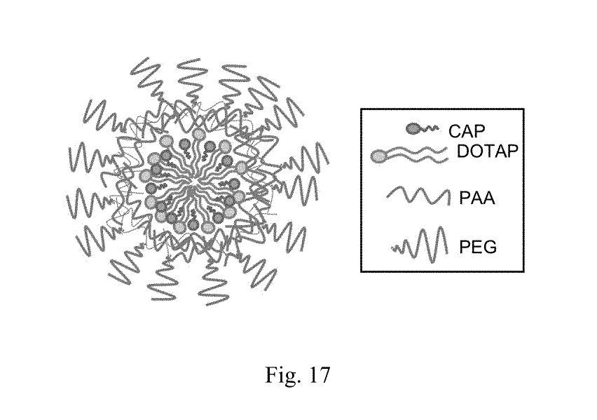

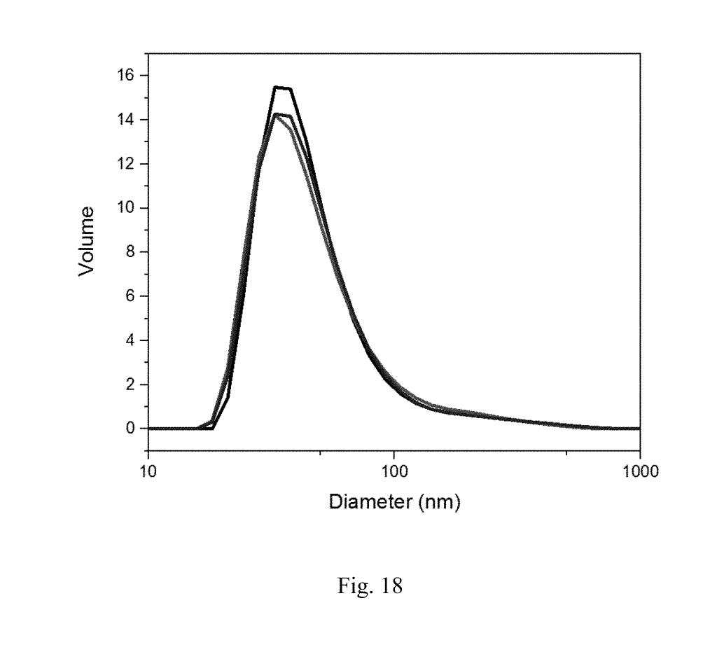

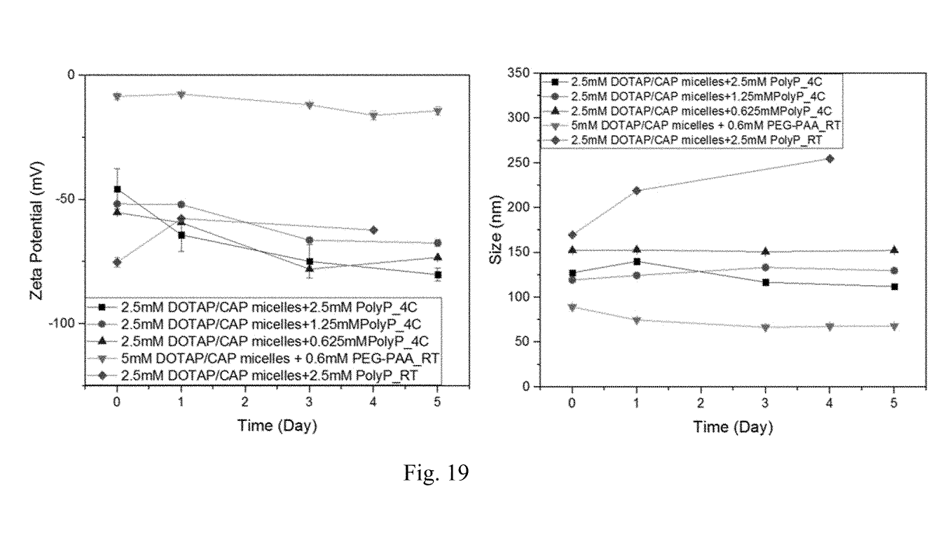

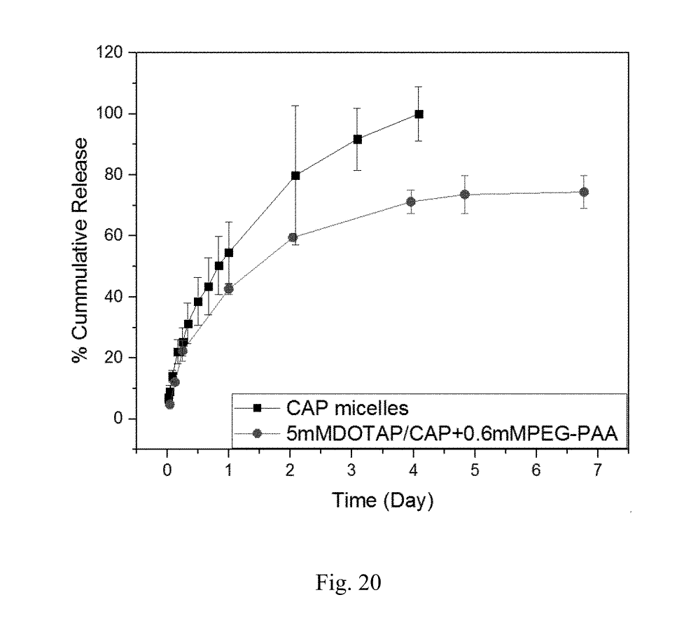

Diblock copolymer PEG-b-PAA was used for steric shielding effects and potential long blood circulation of the nanoparticles. DOTAP-CAP micelles were prepared as described in example 2. PEG-PAA was dissolved in water at 0.6 mM. 500 uL of DOTAP-CAP micelles were mixed with equal volume of PEG-PAA solution. The structure for the PEG-PAA-DOTAP-CAP nanoparticles is shown in FIG. 17. The size distribution of PEG-PAA-DOTAP-CAP nanoparticles is shown in FIG. 18. Zeta-potential and size distribution were monitored for five days at room temperature. As seen in FIG. 19, the zeta potential of PEG-PAA is close to neutral and particles remained stable. The sustained release of CAP from PEG-PAA-DOTAP-CAP nanoparticles over seven days is presented in FIG. 20. Parameters for the PEG-PAA-DOTAP-CAP nanoparticle suspensions is provided in Table 7.

TABLE 7
Information of PEG-PAA-DOTAP-CAP nanoparticle suspension.

| | | | | Average | Zeta |
|---|---|---|---|---|---|
| Concentrations | | | Theoretical drug | diameter | potential |
| DOTAP | CAP | PEG-PAA | loading (wt %) | (nm) | (mV) |
| 5 mM | 5 mM | 0.6 mM | 51.9 | 89.1 | −8.50 |

### Equivalents

Those skilled in the art will recognize, or be able to ascertain using no more than routine experimentation, many equivalents to the specific embodiments of the disclosure described herein. It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A nanoparticle composition comprising a nanoparticle core comprising one or more phospholipids and at least one active ingredient comprising capecitabine or its active metabolites and at least one layer of one or more polymers on a surface of the phospholipids core.

2. The nanoparticle composition of claim 1, wherein one or more of the phospholipids is neutral or positively charged.

3. The nanoparticle composition of claim 1, wherein the one or more phospholipids comprise at least one of 1,2-Didecanoyl-sn-glycero-3-phosphocholine (DDPC), 1,2-dilauroyl-sn-glycero-3-phosphoethanolamine (DLPE), dimyristoyl phosphatidylcholine (DMPC), dimyristoyl phosphatidylglycerol (DMPG), 1,2-dimyristoyl-sn-glycero-3-phosphoethanolamine (DMPE-PEG), 1-palmitoyl-2-myristoyl-sn-glycero-3-phosphocholine (PMPC), 1,2-dipalmitoyl-sn-glycero-3-phospho-(1′-rac-glycerol) (DPPG), 1,2-dipalmitoyl-sn-glycero-3-phosphocholine (DPPC), 1,2-dipalmitoyl-sn-glycero-3-phosphoethanolamine (DPPE-PEG), 1,2-dipalmitoyl-sn-glycero-3-phosphate (sodium salt) (DPPA), 1-palmitoyl-2-oleoyl-sn-glycero-3-phosphocholine (POPC), 1-palmitoyl-2-stearoyl-sn-glycero-3-phosphocholine (PSOC), 1-stearoyl-2-palmitoyl-sn-glycero-3-phosphocholine (SPPC), 1,2-distearoyl-sn-glycero-3-phosphoethanolamine-N-[methoxy(polyethylene glycol)-2000] (DEPE-PEG), 1,2-distearoyl-sn-glycero-3-phosphoethanolamine-N-[dibenzocyclooctyl(polyethylene glycol)-2000] (DSPE-PEG), L-α-phosphatidylcholine (L-α-PC), 1,2-dilinoleoyl-sn-glycero-3-phosphocholine (DLPC), 1,2-dioleoyl-sn-glycero-3-phospho-(1′-rac-glycerol) (DOPG), 1,2-distearoyl-sn-glycero-3-phospho-(1′-rac-glycerol) (DSPG), 1,2-Distearoyl-3-trimethylammonium-propane (DSTAP), 1,2-dioleoyl-3-trimethylammonium-propane (DOTAP), 1,2-dioleoyl-sn-glycero-3-phosphate (DOPA), 1,2-dioleoyl-sn-glycero-3-phosphoethanolamine (DOPE), 1-stearoyl-2-oleoyl-sn-glycero-3-phosphocholine (SOPC), or 1,2-dioleoyl-sn-glycero-3-phosphocholine (DOPC) or combinations thereof.

4. The nanoparticle composition of claim 1, wherein the one or more polymers comprise at least one of poly(lactic-co-glycolic acid) (PLGA) or its PEGylated form PEG-PLGA, polylactic acid (PLA) or its PEGylated form PEG-PLA, polyglycolic acid (PGA) or its PEGylated form PEG-PGA, poly-L-lactide-co-ε-caprolactone (PLCL) or its PEGylated form PEG-PLCL, Hyaluronic acid (HA), polyacrylic acid (PAA) or PEG-PAA, polyphosphate (polyP), poly(acrylic acid-co-maleic acid), poly(butylene succinate), poly(alkyl cyanoacrylate) (PAC) or its PEGylated form PEG-PAC or combinations thereof.

5. The nanoparticle composition of claim 1, further comprising an active agent selected from at least one of an anti-cancer drug, an antibiotic, an antiviral, an antifungal, an antihelminthic, a nutrient, a small molecule, a siRNA, an antioxidant, an antibody, or a radioisotope, or combinations thereof.

6. The nanoparticle composition of claim 1, wherein the one or more lipids comprise 1,2-dipalmitoyl-sn-glycero-3-phosphocholine (DPPC), 1,2-dipalmitoyl-sn-glycero-3-phosphoethanolamine (DPPE-PEG), L-α-phosphatidylcholine (L-α-PC), 1,2-dilinoleoyl-sn-glycero-3-phosphocholine (DLPC), or 1,2-dioleoyl-3-trimethylammonium-propane (DOTAP) or combinations thereof.

7. The nanoparticle composition of claim 1, wherein a molar ratio of the lipid(s) to a PEGylated lipid(s) is about 100:0 to about 50:50.

8. The nanoparticle composition of claim 1, wherein a molar ratio of a saturated lipid(s) to an unsaturated lipid(s) is about 100:0 to about 25:75.

9. The nanoparticle composition of claim 1, wherein a molar ratio of capecitabine to lipid(s) is about 90:10 to about 10:90.

10. The nanoparticle composition of claim 1, wherein a molar ratio of lipid(s) to polymer is about 100:0 to about 10:80.

11. The nanoparticle composition of claim 1, wherein a molar ratio of capecitabine to polymer is about 100:0 to about 10:90.

12. The nanoparticle composition of claim 1, wherein a zeta potential of the nanoparticle is from about −80 mV to about 80 mV.

13. The nanoparticle composition of claim 1, wherein the one or more lipids is 1,2-dipalmitoyl-sn-glycero-3-phosphocholine (DPPC) and 1,2-dipalmitoyl-sn-glycero-3-phosphoethanolamine (DPPE-PEG).

14. The nanoparticle composition of claim 1, wherein the one or more lipids is L-α-phosphatidylcholine (L-α-PC) and 1,2-dipalmitoyl-sn-glycero-3-phosphoethanolamine (DPPE-PEG).

15. The nanoparticle composition of claim 1, wherein the one or more lipids is 1,2-dioleoyl-3-trimethylammonium-propane (DOTAP).

16. The nanoparticle composition of claim 1, wherein the one or more lipids is 1,2-dioleoyl-3-trimethylammonium-propane (DOTAP) and the polymer is polyphosphate (polyP).

17. The nanoparticle composition of claim 1, wherein the one or more lipids is 1,2-dioleoyl-3-trimethylammonium-propane (DOTAP) and the polymer is PEG polyacrylic acid (PAA).

18. The nanoparticle composition of claim 1, wherein a surface of the nanoparticle core is neutral.

19. The nanoparticle composition of claim 1, wherein a surface of the nanoparticle core is positively charged or negatively charged.

20. The nanoparticle composition of claim 1, further comprising at least one targeting agent, wherein the targeting agent selectively targets the nanoparticle to diseased tissue/cells, thereby minimizing whole body dose.

21. The nanoparticle composition of claim 1, further comprising at least one targeting agent, wherein the targeting agent comprises an antibody or functional fragment thereof, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof

22. The nanoparticle composition of claim 1, wherein the nanoparticles have a size of 10 to 200 nm.

23. The nanoparticle composition of claim 1, wherein a drug load of capecitabine is about 2% to about 90% by weight of the composition.

24. The nanoparticle composition of claim 1, wherein the structure of the composition provides sustained release of capecitabine or its active metabolites when provided to a subject.

25. The nanoparticle composition of claim 1, wherein a bioavailability of the active agent is increased, one or more side effects such as nausea, vomiting, dermatitis, bone-marrow depression, cardiotoxicity and diarrhea is reduced, and the active agent is released in a sustained manner.

26. The nanoparticle composition of claim 1, wherein the nanoparticles are adapted for intramuscular, subcutaneous, intravascular, or intravenous administration.

27. A method of forming a nanoparticle composition of claim 1, comprising:
a). forming an organic phase by combining one or more phospholipids, one or more solvents and at least one of capecitabine or its metabolites;
b). forming a lipid aqueous phase by combining one or more targeting agents with water;
c). mixing the organic phase with the aqueous phase, whereby self-assembly of micelles occurs, thereby forming a suspension;
d). spray drying or freeze drying the suspension; and
e). mixing solution with one or more polymers with the micelles, whereby layer-by-layer polymer deposition occurs and wherein the capecitabine or its metabolites nanoparticles provide sustained release of active components when provided to a subject.

28. The method of claim 27, wherein the nanoparticles are produced in a uniform size with uniform physicochemical properties.

29. A method for treating a patient suspected of being afflicted with a disease comprising administering the nanoparticle composition of claim 1.

30. The method of claim 29, wherein administering nanoparticles comprises administering the nanoparticles by intramuscular, subcutaneous, intravascular, or intravenous administration.

31. The method of claim 29, wherein the disease is selected from the group consisting of oncologic, neurologic, and metabolic diseases.

32. The method of claim 29, wherein the disease is selected from the group consisting of Parkinson's disease, Alzheimer's disease, multiple sclerosis, ALS, sequel, behavioral and cognitive disorders, autism spectrum, depression, and neoplastic disease.

33. The method of claim 29, wherein the active agent is released in a sustained manner.

34. A pharmaceutical composition comprising the nanoparticle composition of claim 1 and a pharmaceutically acceptable carrier.

35. The composition of claim 34, wherein administration of the composition to a subject reduces one or more side effects comprising nausea, vomiting, dermatitis, bone-marrow depression, cardiotoxicity or diarrhea or a combination thereof compared to administration of capecitabine that is not formulated in the nanoparticle composition.

36. A method of treating a subject suspected of having cancer comprising:
identifying a subject suspected of having a cancer; and
administering an effective amount of the composition of claim 1 to the subject, wherein administration of the composition reduces one or more side effects comprising nausea, vomiting, dermatitis, bone-marrow depression, cardiotoxicity or diarrhea or a combination thereof when provided to a subject compared to administration of capecitabine that is not formulated in the nanoparticle composition.

37. The method of claim 36, wherein the cancer is a breast cancer, colorectal cancer, or a pancreatic cancer.
